(19) United States
(12) Patent Application Publication
(10) Pub. No.: US 2015/0337351 A1
(43) Pub. Date: Nov. 26, 2015

(54) METHODS OF MICROORGANISM IMMOBILIZATION

(71) Applicant: ACCELERATE DIAGNOSTICS, INC., Tucson, AZ (US)

(72) Inventor: Steven W. Metzger, Tucson, AZ (US)

(21) Appl. No.: 14/720,588

(22) Filed: May 22, 2015

Related U.S. Application Data
(60) Provisional application No. 62/002,746, filed on May 23, 2014.
(60) Provisional application No. 62/058,594, filed on Oct. 1, 2014.

(51) Int. Cl. C12Q 1/06; C12Q 1/08; C12Q 1/10; C12Q 1/04; C12Q 1/14

(52) U.S. Cl. CPC C12Q 1/06; C12Q 1/045; C12Q 1/14; C12Q 1/10; C12Q 1/08

(57) ABSTRACT

The present disclosure is related to methods for immobilizing microorganisms to produce an immobilized microorganism sample for detection with a detection system. Compositions for immobilizing microorganisms are also disclosed.

### CROSS REFERENCE TO RELATED U.S. APPLICATIONS

This non-provisional application claims priority to U.S. Patent Application No. 62/002,746 entitled “Systems and Method of Microorganism Capture, Immobilization, and Detection,” filed May 23, 2014, and to U.S. Patent Application No. 62/058,594 entitled “Methods of Microorganism Immobilization,” filed Oct. 1, 2014, the contents of which are hereby incorporated by reference in their entirety.

### FIELD

This disclosure relates to methods of immobilizing of microorganisms for detection of microorganism information.

### BACKGROUND

Traditional microbiological techniques involve culturing, including automated systems and manual techniques such as broth microdilution or disk diffusion on agar plates. While potentially accurate and clinically relevant, these culturing techniques are time intensive, typically requiring one or more overnight incubations. Clinical microbiological identification and antibiotic susceptibility testing (AST) then can be too slow for critically ill patients whose lives depend on more immediate diagnosis and administration of an effective antibiotic therapy regimen.

Various methods have been developed that can provide rapid and sensitive detection and identification of microbial pathogens, however, many of these methods are not capable of determining whether cells present in the specimen are viable. This is important because mere identification information alone may not be sufficient to direct efficacious therapy, and false positive tests created by non-viable microorganisms can result in critical time lost to misdirected therapeutic efforts. For example, polymicrobial specimens or specimens obtained from patients undergoing antibiotic therapy, may contain non-viable organisms that may be present in a patient. Such organisms may give positive results by various sensitive microorganism identification methods, such as molecular diagnostics methods relying on nucleic acid testing (“NAT”) or immunoassays. Other types of diagnostic testing, such as direct-from-specimen testing using microscopy and other non-destructive methods of detecting growth of individual cells or clones from a sample can enable rapid, culture-free identification, viable cell detection, and AST.

Clinical confidence in diagnostic results, particularly AST results, typically involves sophisticated and time consuming procedures required to ensure sample integrity as a true and accurate biological snapshot of a patient's condition. For example, microorganisms in a specimen must be of a sufficiently large population to support desired data observation and acquisition analyses. Further, sophisticated sample handling procedures are required to facilitate efficiency and throughput potential while maintaining microorganism viability. However, these various procedures can be easily compromised by a variety of conditions. For example, non-microorganism debris easily confounds existing techniques. Likewise, while the need for large populations of microorganism may be required, when too highly concentrated, the microorganisms can produce interference and undesired interactions. Indeed, there are numerous challenges to obtaining accurate observation, identification and susceptibility determinations for microbial samples. Therefore, new methods, compositions, and systems are required to meet these challenges.

### SUMMARY

Provided herein are methods, compositions, and systems relating to immobilizing microorganisms for enhancing the acquisition of microorganism information. Various aspects and embodiments are provided to facilitate data acquisition and tracking of growth from individual microorganisms, while also minimizing interfering effects among and between sample microorganisms or other non-microorganism sample components.

In an aspect, a method of immobilizing microorganisms comprises:

(a) contacting a sample comprising microorganisms with an immobilizing agent to produce a pre-immobilization sample; and

(b) immobilizing the pre-immobilization sample to produce an immobilized sample;

(c) confining a first microorganism to a first location and a second microorganism to a second location in the immobilized sample volume in response to immobilizing the pre-immobilization sample;

(d) wherein the first location and the second location are distinguishable by a system configured to acquire microorganism information.

In another aspect, a method of immobilizing microorganisms comprises:

(a) contacting a sample comprising microorganisms with an immobilizing medium;

(b) electrokinetically introducing the microorganisms into the immobilizing medium to produce an immobilized sample; and

(c) confining a first microorganism to a first location and a second microorganism to a second location in the immobilized sample volume in response to electrokinetically introducing the microorganisms into the immobilizing medium;

(d) wherein the first location and the second location are distinguishable by a system configured to acquire microorganism information.

In an embodiment, the microorganisms are separated from sample debris in response to electrokinetically introducing the microorganisms into the sample.

In another aspect, a method of immobilizing microorganisms comprises:

(a) contacting a sample comprising microorganisms with an absorption medium;

(b) absorbing the sample into the absorption medium to produce a surface-captured sample;

(c) contacting the surface-captured sample with an immobilizing medium to produce a pre-immobilization sample;

(d) immobilizing the pre-immobilization sample; and

(e) confining a first microorganism to a first location and a second microorganism to a second location in response to immobilizing the pre-immobilization sample;

(f) wherein the first location and the second location are distinguishable by a system configured to acquire microorganism information.

In an embodiment, sample debris is separated from the microorganisms in response to absorbing the sample into the absorption medium.

In another aspect, a method of immobilizing microorganisms comprises:

(a) contacting a sample comprising microorganisms with an immobilizing agent to produce a pre-immobilization sample;

(b) contacting the pre-immobilization sample with a biosensor defining a detection space;

(c) inducing a phase change in the pre-immobilization sample to produce an immobilized sample with an immobilized sample volume;

(d) confining a first microorganism to a first location in the immobilized sample volume in response to inducing the phase change;

(e) positioning the biosensor at a first position relative to a detection system;

(f) detecting the first microorganism at the first location;

(g) assigning a first location value and acquiring first microorganism information at a first time in response to detecting the first microorganism;

(h) positioning the biosensor at a second position relative to the detection system;

(i) repositioning the biosensor at the first position and acquiring first microorganism information at a second time based on the first location value; and

(j) determining growth of the first microorganism in response to a change in the first microorganism information from the first time to the second time.

In another aspect, a microorganism immobilizing composition comprises:

(a) an immobilizing agent at an immobilizing agent concentration; and

(b) a nutrient medium at a nutrient medium concentration;

(c) wherein the immobilizing agent is suitable to restrict microorganism movement following addition of the immobilizing agent to a microorganism sample and production of a immobilized microorganism sample; and

(d) wherein the immobilized microorganism sample is compatible with microorganism detection with a detection system.

In various aspects, methods, compositions and systems of immobilizing microorganisms on a surface or in a three dimensional space are provided.

In various aspects, methods, compositions and systems for reducing physical interference between microorganisms are provided.

In various aspects, methods, compositions and systems for preventing a first microorganism from influencing a determination of growth of a second microorganism are provided.

In various aspects, methods, compositions, and systems for combining sample preparation and immobilization are provided.

In various aspects, methods, compositions and systems for maximizing microorganism density in a three-dimensional space are provided.

In various aspects, methods, compositions and systems for minimizing sample debris interference with microorganism detection are provided.

In various aspects, methods, compositions and systems enabling rapid detection of growth by a detection system are provided.

In various aspects, methods, compositions and systems for facilitating detection and tracking of individual microorganisms in a sample comprising a plurality of microorganisms are provided. In various embodiments an immobilizing medium is configured to facilitate acquisition of microorganism information from each individual microorganism over a period of time.

In various aspects, methods and compositions for immobilizing media are provided, where the immobilizing media may be used to restrict microorganism movement, and/or where the immobilizing media is suitable to sustain growth of a plurality of microorganisms, and/or wherein the immobilized media is compatible with a detection system.

Various aspects described herein are useful for determining microorganism information (e.g., data describing a microorganism attribute). More specifically, certain aspects and embodiments described herein facilitate identifying and quantifying microorganism information for individuated microorganism characteristics. The microorganism information may be used to identify and characterize one or more microorganisms in a specimen or sample and/or recommend treatment options based on a microorganism response to a condition (e.g., inclusion or exclusion of one or more antimicrobial agents from a treatment regimen).

Various aspects are useful in identifying individuated microorganisms and evaluating microorganism information and growth under or in response to various conditions. For example, certain microorganism may be exposed to a first condition that stimulates growth (e.g., an increase in temperature) and/or a second condition that inhibits growth (e.g., an antimicrobial agent). As such, various aspects facilitate determining microorganism identification, growth, antimicrobial susceptibility and/or resistance, and providing a variety of analytical outputs based on a multi-variable or multi-factorial analysis.

### BRIEF DESCRIPTION OF THE DRAWINGS/FIGURES

The accompanying drawings, which are incorporated herein and form a part of the specification, illustrate various aspects and embodiments of the present disclosure and, together with the description, further serve to explain the principles of the disclosure and to enable a person skilled in the pertinent art to make and use the disclosed embodiments.

FIGS. 1A-1C illustrate electrokinetic separation of microorganisms and sample debris.

FIGS. 2A-2C illustrate examples of darkfield images of microorganisms in immobilizing media under various conditions.

FIGS. 3A-3F illustrate microorganism growth in a mixed species diffusion assay using immobilized microorganisms.

FIGS. 4A and 4B illustrate cell division rates for various clone densities in an immobilizing medium.

FIG. 5 illustrates growth rate over time for different clone densities in an immobilizing medium.

### DETAILED DESCRIPTION

The detailed description and the accompanying figures and examples describe various aspects and embodiments of the inventions described herein, and are not to be construed as limiting. While these aspects and embodiments are described in sufficient detail to enable those skilled in the art to practice the embodiments, other aspects and embodiments may be realized and changes may be made without departing from the spirit and scope of the present disclosure. Any reference to the singular includes the plural and any reference to more than one component may include a singular component. Any designations such as “first” and “second”, with respect to a device, method or system, is for purposes of convenience and clarity, and should not be construed as limiting. Recitation of multiple aspects and embodiments having stated features is not intended to exclude other aspects and embodiments having additional features or other aspects and embodiments incorporating different combinations of the stated features.

Various aspects and embodiments are also described in terms of systems, methods and compositions throughout. When a particular feature, structure, or characteristic is described in connection with an aspect or embodiment, it is within the knowledge of one skilled in the art to affect such feature, structure, or characteristic in connection with other aspects and embodiments whether or not explicitly described. After reading the description, it will be apparent to one skilled in the relevant art(s) how to implement the disclosure in alternative aspects and embodiments.

Various systems, devices, methods, and the like can be implemented or incorporated herein to perform the intended functions. It should also be noted that the accompanying drawing(s)/figure(s) referred to herein may not be drawn to scale and in that regard, the drawing(s)/figure(s) should not be construed as limiting. Finally, although the present disclosure can be described in connection with various principles and beliefs, the present disclosure is not intended to be bound by any particular theory.

### DEFINITIONS

The term “medium” (and plural “media”) as used herein means a fluid, gel, or solid designed to support microorganisms or cells. In some cases, the medium is designed to support the viability or growth of a microorganism or cell for a period of time. The term “medium” also includes “pre-immobilizing medium,” “immobilizing medium,” “growth medium,” “culture medium” and other similar terms that may be used to refer a composition suitable to support microorganism or cells. The term “medium” can also refer to the physical medium comprised in a biological sample containing microorganisms.

The terms “immobilize,” “immobilizing” and “immobilization” are used herein to denote physically restricting the movement of an object, such as a microorganism. The term “immobilizing” includes restriction in a relative sense, rather than an absolute sense, and “immobilizing” can be used to describe, for example, the properties of a medium to impose a higher resistance to the movement of a particle within the medium as compared to another physical environment or sample medium.

The term “immobilizing agent” as used herein means one or more agents that may be added to a medium to provide the medium with an immobilizing property.

The term “immobilizing medium” as used herein means a medium configured to facilitate immobilization of a microorganism.

The term “immobilized sample” as used herein means an immobilizing medium comprising immobilized microorganisms.

The term “confine” as used herein means to restrict an object to a location in space over a period of time. An object may be confined to a discrete physical or theoretical location. The term “confine” can include any of a point location (e.g., a discrete location to which an object is confined, such as a point no larger than the object itself, or the point at which the center of mass of the object is essentially fixed); or, a volume of space (e.g., a physical or theoretical region of space defined by a boundary beyond which the object (or objects derived from the original object, such as progeny cells, particulate debris, secreted macromolecules, metabolic byproducts, and the like) cannot move or is probabilistically unlikely to move). A boundary may be a discrete physical boundary, or a boundary may be a theoretical (probabilistic) boundary.

The term “microorganism” as used herein means a microscopic organism, such as a member of one or more of the following classes: bacteria, fungi, algae, and protozoa. A microorganism can include a single cell, a plurality of clonally derived cells (i.e., a clone), or a multicellular organism. It can also include viruses, prions, or other pathogens. In various aspects and embodiments, a microorganism comprises a human or animal pathogen, such as a bacterium. With respect to bacteria, a microorganism can include any genus, species, or strain, subtype, or genetic variant, including those well established in the medical field as well as any novel bacteria and variants that emerge from time to time.

The term “plurality of microorganisms” as used herein means more than one microscopic organism. When describing a sample comprising a plurality of bacterial microorganisms, a “plurality of microorganisms” means more than one colony forming unit (“CFU”).

The terms “sample” or “microorganism sample” as used herein refer to any physical medium that comprises a microorganism. Generally, a “sample” or “microorganism sample” comprises microorganisms from a biological or clinical sample or specimen, whether directly from the source or further processed. A sample will frequently be a liquid sample having a volume.

The terms “biological/clinical sample,” “biological/clinical specimen” as used herein mean a sample derived from a biological organism such as a human or an animal.

The term “polymicrobial sample” as used herein means a sample comprising two or more microorganisms that are different, such as different genera, different species, different strains, different subtypes, genetic variants, or the like.

The term “sample microorganism concentration” as used herein refers to a particular density of microorganisms in a sample, such as may be expressed as a number of microorganisms or CFU per unit volume of sample.

The term “sample composition” as used herein means the physical and/or chemical constituents of a sample, including microorganisms, non-microorganism cells, particulate debris, macromolecules, small molecules, ions, and the like.

The term “pre-immobilization sample” as used herein means a sample that has not been immobilized. A pre-immobilization sample may comprise an immobilizing agent that has not yet immobilized the sample, such as in the case of a gelling agent that has not undergone a phase change to solidify or gel the sample, or a viscous solution that has not yet been intermixed through the sample.

The term “pre-immobilization sample composition” as used herein means the composition of a sample, relative to any or all of its constituent components, prior to any immobilization of the sample. A pre-immobilization sample composition may include components that have been added to a sample to adjust some physical or chemical parameter of the sample.

The term “pre-immobilization sample concentration” as used herein means the concentration of microorganisms in a sample prior to immobilization of the sample.

The term “sample debris” as used herein means non-microorganism sample constituents, such as non-microorganism cells (e.g., blood cells from a blood culture sample), cellular debris from disrupted microorganism or non-microorganism cells, macromolecules such as proteins or polypeptides, nucleic acids, polysaccharides, lipids, and other biomolecules or non-biomolecule macromolecules, and the like. Sample debris may be used to refer to particulate matter suspended in a solution, or sample debris may further include small molecule solutes.

The term “immobilized sample properties” as used herein means the physical properties or attributes of an immobilized sample medium following immobilization. Physical properties of an immobilized sample medium may include any of a variety of subjectively- or objectively-measurable attributes, including the viscosity of the immobilized sample, the opacity of the immobilized sample to a particular wavelength of light, etc.

The term “immobilized sample volume” as used herein means the area of space occupied by an immobilized sample, as defined by the boundaries of the immobilized sample volume. In various embodiments, the “immobilized sample volume” will be defined or partially defined by a detection device, such as a flowcell of a microfluidic detection device, a microcapillary tube, a microwell or microcuvette, a slide, or similar device. An “immobilized sample volume” can also be partially defined by a physical boundary of the immobilizing medium of the immobilized sample that is not in contact with a detection device.

The term “detection system” as used herein includes any of various suitable systems, devices, methods and compositions configured to perform microorganism detection, identification, or related analyses, including determination of growth, susceptibility to antimicrobial agents, and the like.

The term “identification” as used herein means the determination of the identity of a microorganism, such as a determination of the genus, species, strain, genotype, or other categorical descriptor that may be applied to describe a microorganism.

The term “microorganism detection” as used herein means detection of a microorganism or acquisition of microorganism information.

The term “microorganism information” as used herein means information or data relating to an attribute of a microorganism that may be detected or measured by a detection system.

The term “individuated” as used herein means an object that is physically distinct from another object (i.e., a discrete physical unit) and/or distinguishable from any other object by a detection system at some point in time in a detection process.

The term “location” as used herein can refer to a point in space, including two dimensional or three-dimensional space, or can refer to a defined volume of space, such as a spherical volume defined in relation to a central point.

The term “growth” as used herein means any measurable change of an attribute of a microorganism over a period of time. Growth can be used to describe any change, regardless of whether the change is positive or negative, as well as a lack of a net measureable change over a period of time. Examples of measureable attributes include cell or clone mass, cell divisions (e.g., binary fission events or cell doubling resulting in the production of daughter cells), cell number, cell metabolism products, cell morphology changes (including, for example, filamentation), or any other experimentally observable attribute associated with a microorganism. Detection of growth does not require that cell division be observed. Growth can be used to refer to changes associated with a single microorganism (i.e., a single cell, colony, or clone), as well as a net or collective change for a plurality of microorganisms.

### Immobilization Methods and Compositions

Microorganism Samples

In various aspects, a microorganism sample is obtained. A microorganism subjected to an immobilization method can include both clinical and non-clinical samples in which microorganisms are known or suspected to be present. A microorganism sample can comprise one or a plurality of microorganisms. The amount of a microorganism sample used in the various methods disclosed herein may be based on the source of the sample and/or the nature of the sample. Samples may be obtained and/or prepared by any of a number of methods known to a person of ordinary skill in the art. In various embodiments, samples obtained from various sources may require little or no preparation prior to processing by the methods disclosed herein.

In various embodiments, a microorganism sample may be a biological sample, including both clinical specimens and research samples. Biological samples can include any type of sample that may be obtained from a human or animal patient or subject, such as a blood sample, a blood fraction, serum, plasma, synovial fluid, sputum, saliva, urine, feces, semen, vaginal secretions, cerebrospinal fluid, gastrointestinal system fluid, tissue homogenates, bone marrow aspirates, swabs and swab rinsates, other bodily fluids, and the like. In various embodiments, a clinical sample may be cultured and the cultured sample, such as a blood culture, can comprise a microorganism sample.

Non-clinical samples that may be used can include, but are not limited to, food products, beverages, pharmaceuticals, cosmetics, water (e.g., drinking water, non-potable water, and waste water), seawater ballasts, air, soil, sewage, plant material (e.g., seeds, leaves, stems, roots, flowers, fruit), blood products (e.g., platelets, serum, plasma, white blood cell fractions, etc.), donor organ or tissue samples, biowarfare samples, and the like. Samples can also be used for real-time testing to monitor contamination levels, process control, quality control, and the like in industrial settings. In another embodiment, the non-clinical sample can be cultured, and a culture sample used.

In various embodiments, a sample may be a cleaned-up sample substantially free of interfering, non-microorganism sample debris or other sample components. Similarly, a microorganism sample can comprise microorganisms that have been subjected to a surface capture step such as electrokinetic concentration (“EKC”) or that have been absorbed onto a surface of a preparative gel, such as following gel electrofiltration or other similar sample preparation steps.

In various other embodiments, a sample need not be cleaned-up or subject to any type of sample purification prior to introduction to immobilization and detection, and instead a sample may be subjected to an immobilization method directly following collection or following a sample concentration step. A sample introduced to an immobilization method of the present disclosure may comprise viable microorganisms along with sample debris, including cellular debris particles, (such as microorganism debris, blood cells or other non-microorganism cells or cellular debris from the specimen, as well as other small molecules and macromolecules that may interfere with microorganism detection, identification, and/or AST analysis. These and other characteristics of the sample that can influence the success of a microorganism detection method may be evaluated and/or addressed in various steps upstream of immobilization and detection, or they may be addressed in the course of an immobilization and detection method.

Immobilizing Agents and Immobilizing Media

In various aspects and embodiments, the microorganism sample is contacted with an immobilizing agent to produce a pre-immobilization sample. In various embodiments, contacting a microorganism sample with an immobilizing agent comprises adding a chemical or physical agent to a microorganism sample to provide an increase in the resistance of sample medium to particle movement. The increase in the resistance of the sample medium to particle movement may occur following an immobilizing step, described in greater detail below, that produces an immobilized sample or an immobilizing medium.

Various immobilizing agents traditionally used in microbiological media are well known and may be compatible with the methods, compositions and systems herein and serve as immobilizing agents suitable to produce an immobilized sample or an immobilizing medium in accordance with various embodiments. In various embodiments, an immobilized sample or immobilizing medium may comprise a gel-immobilizing agent (i.e., an immobilizing agent that may confer gel-like properties to an immobilized sample or an immobilizing medium). In various other embodiments, an immobilized sample or immobilizing medium may comprise a immobilizing agent that produces a viscous fluid or increases the viscosity of a fluid to which it is added. Any agent suitable to provide immobilization of a microorganism as defined herein is within the scope of the present disclosure. Immobilizing agents, immobilizing media, and the general properties of these, are described in greater detail below.

In various aspects, an immobilizing agent is a gel-immobilizing agent. A gel-immobilizing agent is an immobilizing agent suitable to provide a solid three-dimensional network extending throughout the volume of a fluid medium extender. The fluid phase extender is a solution that expands the volume of the gel-immobilizing medium or sample. Thus, a gel-immobilized sample may be diphasic, comprising the solid phase network and the fluid phase extender (also referred to simply as the fluid phase or fluid medium (of the gel)). The fluid phase may comprise water (e.g., hydrogels and aquagels) or air (aerogels), along with any solutes and other suspended components that may be present in the fluid phase. For example, in various embodiments, the fluid phase of the gel may comprise the immobilized microorganisms. In various embodiments, the internal three-dimensional structure of a gel serves as a scaffolding and/or boundary network providing for or contributing to the immobilization of the microorganisms in the fluid medium of the gel.

In various embodiments, the solid phase three-dimensional network of a gel may comprise a nonfluid colloidal network or a polymer network. Either network may comprise physical and/or chemical bonds aggregating or crosslinking the network elements. A nonfluid colloidal network may comprise lamellar structures or particulate disordered structures, including globular and fibrillar protein gels. A polymer network can include any of a covalent polymer network; a polymer network bonded by physical aggregation of polymer chains producing network junction points, such as by hydrogen bonds, crystallization, helix formation, complexation, and the like; or a polymer network formed through glassy junction points, such as with block copolymers. Any gelling agent or gel material that provides material properties compatible with microorganism immobilization and detection may be used as an immobilizing agent in accordance with the present disclosure.

Suitable gel-type immobilizing agents include natural and synthetic gelling agents. Examples of natural gelling agents include, but are not limited to, agar, gellan gum, guar gum, agarose, carrageenans, cassava starch, zeins, gelatin, alginates, collagen, fibrin, proteoglycans, elastin, hyaluronic acid, glycoproteins such as fibronectin and laminin, and the like. Examples of synthetic gelling agents include, but are not limited to, methyl cellulose, vinylpyrrolidone, 2-methyl-5-vinylpyridine, acrylates, vinyl alcohol, vinyl pyridine, vinyl pyridine-styrene, and the like, along with numerous variations and derivatives of the same. Various types of nanoparticles and carbon nanotubes may also comprise a gel or gel-like diphasic system and be used as an immobilizing agent in accordance with various embodiments. Any chemical or physical agent known or hereinafter discovered that may be added to a fluid medium and is suitable to produce a solid physical network structure throughout at least a portion of the medium and provide material properties that facilitate immobilization of one or more microorganisms within a sample may be used as an immobilizing agent in accordance with the present disclosure.

In various aspects, an immobilized sample or an immobilizing medium can also comprise an immobilizing agent that produces a viscous fluid immobilized sample or immobilizing medium. In various embodiments, addition of a viscosity-increasing immobilizing agent to a microorganism sample may restrict movement of a microorganism within the sample within the meaning of the terms “immobilizing agent” and “immobilizing medium,” as used herein.

Any of a number of viscosity-increasing immobilization agents may be used, including, for example, polysaccharides such as starches, gums, and pectins, including agar, carrageenan, alginates, levan, guar gum, xanthan gum; polysaccharide derivatives; cellulose ethers (including, for example, methyl cellulose, ethyl cellulose, and other cellulose ether polymers and derivatives); polyvinyl alcohol; polyoxyalkylene alkyl ether; polypropylene glycol; glycerol; poly-γ-glutamic acid; and the like, in particular those compatible with microorganism growth and/or detection.

Compatibility of Immobilizing Agents and Immobilizing Media with Growth and Detection

In various aspects and embodiments, an immobilizing agent and/or immobilizing medium is selected to provide material properties compatible with homeostasis and growth of a microorganism. In various embodiments, an immobilization method is performed to facilitate detection of microorganism growth, and an immobilizing medium is suitable to support viability and growth of immobilized microorganisms. Thus, in various embodiments, the immobilizing agent selected is suitable to maintain microorganism viability throughout an immobilizing process, including through steps of contacting a microorganism sample with the immobilizing agent, immobilizing a pre-immobilization sample, and detecting growth of a microorganism. Stated differently, in various embodiments, the material properties of the immobilizing agent do not substantially affect homeostasis of a microorganism or a growth rate of microorganism as compared to a non-immobilized control sample.

In various embodiments, an immobilizing agent and/or medium is selected to provide material properties compatible with microorganism detection. In various embodiments and as described in greater detail below, a detection system and method is used to obtain microorganism information for immobilized microorganisms. A suitable immobilized sample may be configured by appropriate selection of an immobilizing agent having material properties compatible with a detection system or a method for acquisition of microorganism information. In various embodiments, suitable immobilizing agents include those with material properties that facilitate production of an optically transparent immobilizing medium. In certain embodiments, the immobilizing agent is selected to provide an immobilizing medium compatible with use of optical detection systems and methods for acquisition of microorganism information, such as by brightfield or darkfield microscopy. In other embodiments, an immobilizing agent is selected to provide an immobilizing medium compatible with fluorescence microscopy. In still other embodiments, microorganism information may be obtained using non-optical detection systems, and optical transparency of the immobilizing medium is not required for microorganism detection and acquisition of microorganism information. In such embodiments, an immobilizing agent and/or immobilizing medium may be selected to provide properties that are compatible with the non-optical detection method applied for acquisition of microorganism information.

Ability of Immobilizing Media to Produce Local Microenvironments

In various aspects, an immobilizing medium may be configured to produce one or more local microenvironments within the immobilizing medium. An immobilizing medium can comprise a local microenvironment based on the relative continuity of the fluid phase of the medium and/or the viscosity of the medium. In various embodiments, an immobilizing agent may be selected to provide an immobilizing medium with a fluid phase that may be relatively continuous and non-viscous. In other embodiments, an immobilizing agent may be selected to provide an immobilizing medium that is relatively discontinuous and/or viscous.

In various aspects and embodiments, the relative continuity and/or viscosity of the fluid phase of an immobilizing medium can influence a rate of diffusion of a solute or an object suspended in the fluid phase. In various embodiments, an immobilizing agent may be selected to provide an immobilizing medium configured to provide a desired effect with respect to the rate of diffusion of an object such as ion, small molecule, macromolecule, or other particle in the immobilizing medium.

In various embodiments, the rate of diffusion of an object such as ion, small molecule, macromolecule, or other particle in a medium may be quantified. For example, the rate of diffusion of an object in terms of a distance travelled per unit time may be measured and expressed relative to the rate of diffusion of the same object in a reference medium such as water or any other suitable liquid medium under the same physical conditions. In various embodiments, the properties of an immobilizing medium may be configured to provide a desired rate of diffusion of one or more components of an immobilized sample relative to the rate of diffusion in a reference medium. For example, the gel strength may be adjusted to provide a particular diffusion rate for a particular object or molecule with respect to a medium comprising the same fluid medium component without the immobilizing agent. In various embodiments, the gel strength may be expressed as providing about a 50% reduction in the diffusion rate, about a 70% reduction in the diffusion rate, about a 90% reduction in diffusion rate, or about a 95% reduction in the diffusion rate of an object. Any object, whether a solute or a suspended particle, may be used as a reference compound relative to which the capacity of an immobilizing medium to restrict diffusion may be expressed.

In various aspects and embodiments, an immobilizing agent may be selected to provide an immobilizing medium with a continuous or non-viscous fluid phase and/or a relatively high rate of diffusion with reference to an object. In various embodiments, a gel-immobilizing agent may be selected to provide an immobilizing medium with a continuous fluid phase. For example, a gel-immobilizing agent may be selected to provide an immobilizing medium comprising pores or void spaces in the solid network structure of the gel. The pores or void spaces may be sufficiently sized and distributed throughout the gel to accommodate diffusion of solutes and objects smaller than microorganisms while restricting the movement of microorganism-sized objects. Likewise the composition of the fluid phase extender can also influence a rate of diffusion within a gel-immobilizing medium. The chemical composition of the fluid phase of a gel, including the solvent, solutes and suspended components, and their concentration and densities may influence a rate of diffusion of a first molecule solute of the fluid medium in the gel.

The fluid phase extender of an immobilizing medium may be sufficiently continuous or non-viscous to relatively free diffusion of solutes or other particles throughout the fluid phase of the medium. In various embodiments, free and/or rapid diffusion of small molecules within an immobilized sample may be desired. For example, an immobilizing medium may be configured to permit diffusion of small molecules, nutrients, ions and other chemical components required by a microorganism for homeostasis and/or growth throughout the fluid phase of the medium, while still providing immobilization of microorganisms. Likewise, in various embodiments, bulk flow of a fluid medium into and/or through a gel-immobilizing medium may be desired, and a gel-immobilizing medium may be configured to accommodate exchange of a fluid medium without disruption of the gel's solid physical network or immobilization of microorganisms in the gel.

In various aspects, limited or no diffusion of various molecules and/or solutes in an immobilizing medium may be desired. An immobilizing agent may be selected to provide an immobilizing medium configured to limit diffusion of macromolecules, small molecules, ions or other solutes within the medium, as described further below.

In various embodiments, the rate of diffusion of small molecules in an immobilizing medium may be limited or controlled by the composition of the immobilizing medium. The choice of immobilizing agent, the immobilizing agent concentration, the composition of the fluid medium, communication or lack of communication of the fluid medium with an external fluid source, other physical environmental parameters, and the characteristics of the small molecule itself may influence the rate of diffusion of a small molecule in an immobilizing medium. In various embodiments, these and other variables may be manipulated to provide a desired level of control of the diffusion of one or more small molecules that may be involved in microorganism metabolism, AST or other assays directed toward determining microorganism growth and/or the response of a microorganism to a condition.

In various embodiments, an immobilizing agent can be selected to provide an immobilizing medium configured with a matrix of bounded domains within the immobilizing medium. For example, a gel-immobilizing agent can provide an immobilizing medium with a solid physical network providing bounded domains suitable to confine and compartmentalize a microorganism. The characteristics of the bounded physical domains may be dependent on the nature and strength of the immobilizing agent and the network it produces. In various embodiments, the bounded domains of the network may have differing degrees of porosity, such as in a manner dependent on the concentration of the immobilizing agent in the immobilized sample or immobilizing medium. For example, at a low end of an immobilization agent concentration range, the porosity of a gel-immobilizing medium may be very high and insufficient to immobilize microorganisms. At the other end of the range, the porosity of a gel may be sufficiently low, for example, to provide a matrix of bounded physical domains suitable to completely physically confine and compartmentalize one or more sample microorganisms in a discrete compartment that is not in fluid communication with a neighboring compartment. For example, in a gel-immobilizing medium comprising a high gel strength, the physical network of the gel may be sufficiently non-porous to render the fluid extender discontinuous within a detection device chamber containing the medium, and the fluid medium in such an embodiment may not be in fluid communication throughout the entire volume of the detection device chamber, thereby limiting diffusion of small molecules in the immobilizing medium.

In various aspects, a viscous fluid immobilized sample can provide local microenvironments within an immobilized sample. In various embodiments, the increase in viscosity of an immobilized sample can restrict the diffusion of small molecules, secreted enzymes, and the like, in a manner such as that described by the Stokes-Einstein equation,

$$D = \frac{k BT}{6 π η r}$$

where D is the self-diffusion coefficient of an ion (or other analogous particle), kB is the Boltzmann constant, T is the temperature, r is the radius of the diffusing particle, and q is the bulk viscosity of the solution. Increasing the viscosity of the immobilized sample may thereby contribute to creation or maintenance of a discrete microenvironment that may be associated with a microorganism confined by the immobilized medium.

In various embodiments, an immobilized sample with a discontinuous fluid medium and discrete local microenvironments may be produced using other methods, such as by creating gel or fluid microdroplets. Each microdroplet may be defined by a boundary comprised of an interface with another fluid, a membrane, or the like. Each microdroplet thereby comprises a discrete volume, wherein the contents of each microdroplet are not in fluid communication with the contents of adjacent microdroplets, while the immobilized sample volume contained in a biosensor sample chamber comprises a plurality of microdroplets.

In various aspects, an immobilizing medium may be suitable to provide a discrete local microenvironment in the vicinity of each immobilized microorganism. For example, an immobilizing medium may provide a first microenvironment at a first location associated with a first microorganism and a second microenvironment at a second location associated with a second microorganism. In various embodiments, an immobilizing medium may be suitable to restrict diffusion of macromolecules or macromolecular structures, while permitting substantially uniform exposure of each microorganism immobilized in the medium to various other small molecules, nutrients, and ions. For example, a gel-immobilizing medium may be suitable to restrict diffusion of secreted or extracellular proteins, glycoproteins, enzymes, virulence factors, exotoxins, metabolic waste products, nucleic acids, or released vesicles or other macromolecular structures between a first location and a second location in the immobilization medium adjacent to the first location by providing a physical boundary of the solid network of the gel immobilization agent between the first location and the adjacent second location, while permitting diffusion of ions and small molecules. In various other embodiments, an immobilizing medium may be suitable to substantially or completely confine microorganisms in a bounded domain that is not in fluid communication with adjacent bounded domains (i.e., a first location adjacent to a second location), thereby providing a local microenvironment for which diffusion of both macromolecules and small molecules is substantially restricted.

In various embodiments, creation of microenvironments and restriction of secreted or extracellular substances produced by an immobilized microorganism can facilitate resolution of polymicrobial samples by reducing instances in which microorganism information for a first microorganism may be influenced by secreted or extracellular substances produced by a second microorganism.

### Pre-Immobilization Sample Preparation

In various aspects and embodiments, contacting a microorganism sample with an immobilizing agent comprises adding an immobilizing agent to a microorganism. In accordance with various embodiments, contacting a microorganism sample with an immobilizing agent produces a pre-immobilization sample. In a pre-immobilization sample, the sample microorganisms are not yet immobilized pending an immobilizing step being performed on the pre-immobilization sample, as described in greater detail below.

In various embodiments, an immobilizing agent may be added to a microorganism sample in a solid form. For example, in various embodiments, contacting a microorganism sample with an immobilizing agent may be performed by adding an immobilizing agent to the microorganism sample in a powder form.

In various embodiments, an immobilizing agent can be added to a microorganism sample in a liquid form as a solution comprising an immobilizing agent. An immobilizing agent in a liquid form is referred to as an immobilizing agent solution. An immobilizing agent solution can comprise the immobilizing agent at an immobilizing agent concentration. The immobilizing agent concentration in the immobilizing agent solution may be configured to provide a final immobilizing agent concentration in the pre-immobilization sample and/or the immobilized sample that is suitable to provide desired immobilized sample properties following immobilization, such as an ability to confine a first microorganism and a second microorganism present in the sample to a first location and a second location, respectively, in the immobilized sample, as described in greater detail below.

In various embodiments, an immobilizing agent solution added to a sample to produce a pre-immobilization sample may further comprise other components not directly related to immobilization of the sample. For example, an immobilizing agent solution may comprise a nutrient component, a buffer component, an antimicrobial agent, or other component. In various embodiments, an immobilizing agent solution may comprise a nutrient component at a nutrient component concentration. For example, and immobilizing agent solution may comprise Mueller-Hinton broth at a particular concentration along with agar or agarose as an immobilizing agent at an immobilizing agent concentration (i.e., Mueller-Hinton agar, “MHA”). The nutrient component concentration and/or the immobilizing agent concentration in the immobilizing agent solution may be configured to provide a desired immobilizing medium final nutrient concentration and/or an immobilizing medium final immobilizing agent concentration. Likewise, in various embodiments wherein antimicrobial susceptibility testing will be performed, an antibiotic agent may be added to the immobilizing agent solution at an antibiotic agent concentration to provide an immobilizing medium with an antibiotic agent concentration suitable to perform susceptibility testing.

A pre-immobilization sample can have a pre-immobilization sample concentration with respect to the density of the microorganisms therein. A pre-immobilization sample can also have a pre-immobilization sample composition with respect to any of the components of the pre-immobilization sample following contacting the microorganism sample with the immobilizing agent.

In various embodiments, a pre-immobilization sample concentration or composition may be adjusted relative to various physical or chemical parameters prior to immobilizing the sample so that the immobilized sample subject to microorganism detection will have various desired properties, such as a suitable microorganism density, debris density, chemical composition (i.e., nutrient medium concentration, antibiotic concentration, etc.), and the like. A pre-immobilization sample may be adjusted based on measured or assumed properties of a microorganism sample, or a pre-immobilization sample may be adjusted based on properties of the pre-immobilization sample itself.

In various embodiments, the components of a pre-immobilization sample may be adjusted prior to producing the pre-immobilization sample. For example, the properties of a microorganism sample may be adjusted prior to contacting with an immobilizing agent, such as by addition of a microbiological nutrient medium independently of addition of the immobilizing agent. In various embodiments, a pre-immobilization sample may be produced by contacting a microorganism sample with an immobilizing agent, and the pre-immobilization sample may be subjected to adjustment prior to performing immobilization of the sample. Examples of adjustments that may be made to a pre-immobilization sample are described further below.

In various embodiments, a sample or a pre-immobilization sample may be adjusted in response to a characteristic of the sample. For example, at least one of a pre-immobilization sample microorganism concentration and a pre-immobilization sample composition may be adjusted in response to the sample microorganism concentration, a sample debris concentration, and a sample composition. In various embodiments, a microorganism concentration of a pre-immobilization sample may be diluted or concentrated in response to the sample microorganism concentration to provide an immobilized sample concentration suitable to facilitate distinguishing the immobilized microorganisms within the time frame of an assay, such as an identification, growth detection, or AST assay. A pre-immobilization sample may similarly be diluted to adjust the concentration in response to the sample debris concentration.

In various embodiments, the pre-immobilization sample composition may be adjusted. For example, in various embodiments, a pre-immobilization sample composition may be adjusted by adding a protease, a detergent, or other component to the pre-immobilization sample to reduce the sample debris concentration and/or a sample debris size.

Likewise, in various embodiments, other components may be added in response to other aspects of a pre-immobilization sample composition aside from the sample debris. For example, in various embodiments, a pre-immobilization sample composition may be adjusted with respect to pH, ion concentration, nutrient concentration, and the like.

In various embodiments, an immobilizing agent concentration may be adjusted to provide a suitable gel strength. A high gel strength may be associated with low porosity and/or large bounded domain size, and a low gel strength may be associated with high porosity and/or a small bounded domain size. For example, in various embodiments, gel strength may be optimized based on the ability to confine a particular microorganism to an area in space over the time frame of a particular assay, or gel strength may be optimized to confine the growth of a microorganism (i.e., a CFU) to a certain clone size in the time frame of an assay. In various embodiments, the gel strength of an immobilizing medium may be suitable to confine the growth of a clone to a diameter of less than about 100 μm, or less than about 50 μm, or less than about 25 μm, or less than about 10 μm, or less than about 5 μm, within a certain assay condition and time period.

In various embodiments, the gel strength or gel properties of an immobilizing medium may also be manipulated to optimize other parameters of the gel, such as the ability of the gel to restrict the diffusion of small molecules, enzymes, debris, or other non-microorganism particles. For example, gel strength may be adjusted for a particular sample type to provide immobilization of sample microorganisms while permitting migration of sample debris in an applied electrical field. In other embodiments, gel strength may be adjusted to reduce the diffusion of bacterial secreted toxins or enzymes, thereby facilitating the creation of discrete chemical microenvironments associated with each immobilized microorganism initially immobilized (i.e., each CFU) and reducing inter-colony antagonistic effects or interferences (as described above).

In various embodiments, a pre-immobilization sample may be configured to be fluidly transferrable into a detection device such as a microvolume detection device chamber in a pre-immobilization sample condition. A pre-immobilization sample condition may be, for example, a temperature of a pre-immobilization sample that is above a temperature at which a phase change of a gel-immobilizing agent occurs.

### Immobilizing a Pre-Immobilization Sample

In various aspects, microorganisms from a microorganism sample are immobilized in an immobilizing medium by an immobilizing step. As defined above, the terms “immobilize,” “immobilization,” and “immobilizing” mean to restrict the relative movement or migration of a microorganism. Restriction of the relative movement or migration of a microorganism effectively produces confinement of the microorganism to a discrete physical or theoretical location in the immobilizing medium. Thus, a microorganism is confined in an immobilization medium in response to immobilizing the microorganism, resulting in establishment and maintenance of an association with a physical location in the immobilizing medium over a period of time, such as a period of time necessary to determine whether a microorganism is growing or a microorganism response to a condition. For example, a first microorganism may be confined to a first location in the immobilized sample, and a second microorganism may be confined to a second location in the immobilized sample, as described in greater detail below.

In various embodiments, immobilizing microorganisms and/or a pre-immobilization sample comprises an additional process step following production of the pre-immobilization sample. Immobilizing microorganisms and/or a pre-immobilization sample may be performed by various methods. In various embodiments, immobilizing a pre-immobilization sample can comprise mixing the immobilizing agent with the pre-immobilization sample after contacting the sample with the immobilizing agent. For example, a fluid immobilizing agent that increases the viscosity of the sample to produce an immobilized sample may be mixed with the microorganism sample following the contacting step in order to produce and immobilized sample. In various other embodiments, immobilizing a microorganism sample can comprise inducing a phase change for an immobilizing agent in a pre-immobilization sample. For example, in various embodiments, a gel-immobilizing agent in a pre-immobilization sample may undergo a phase change to form a solid physical network, thereby forming an immobilizing medium and immobilizing the microorganism in the pre-immobilization sample. In various other aspects and embodiments, a method of immobilizing microorganisms can comprise contacting a microorganism sample with an immobilizing medium and/or introducing the microorganisms into the immobilizing medium to produce an immobilized sample.

In various embodiments, at least one of the steps of contacting and immobilizing microorganism sample can be optimized to reduce an incidence rate of a false negative microorganism detection event for a biological sample. For example, and as described in greater detail herein, various parameters such as immobilizing agent selection, immobilizing agent temperature, immobilizing agent or pre-immobilization sample composition, pre-immobilization sample handling, and the like may be experimentally optimized relative to test biological samples comprising a known microorganism composition to ensure that one of the steps of contacting the microorganism sample with an immobilizing agent or immobilizing the pre-immobilization sample is compatible with obtaining an accurate determination of the presence and viability of the microorganisms in the biological sample.

Various methods of immobilizing microorganisms are described in greater detail below.

Immobilization by Inducing a Phase Change

In various embodiments, the physical form of an immobilizing agent in a pre-immobilization sample may be induced to change from one physical phase to another physical phase in response to a phase change condition (i.e., an inducible phase change). For example, a gel-immobilizing agent in a pre-immobilization sample may be induced to change from a liquid phase to a solid phase. In various embodiments, inducing a phase change of the immobilizing agent produces a change from a liquid pre-immobilization sample to a solid or gel immobilized sample following the immobilizing step due to formation of a solid three-dimensional network structure by the immobilizing agent. For some immobilizing agents, a phase change of the immobilizing agent may be a function of the temperature of the immobilizing agent. For example, a phase change of an agar-immobilizing agent from a liquid form to a gel or solid form may be induced in response to cooling a pre-immobilization sample. In various embodiments, cross-linking of polymer chains or formation of junctions in the network structure may occur in response to addition of a chemical agent (i.e., a cross-linker or other catalyst), photo-reactive cross-linking, exposure to a magnetic field (i.e., magnetorheological fluids), and other chemical or physical mechanisms. In accordance with various embodiments, formation of a solid, three-dimensional network structure in a gel medium may be precisely controlled for various immobilizing media by an operator based on addition of an energy or chemical input to produce an immobilizing medium with a desired property at a desired point in time.

Immobilization by Mixing

In various other embodiments, immobilizing a sample may comprise a step other than inducing a phase change in an immobilizing agent. In various embodiments wherein the immobilizing agent produces an increase in the viscosity of an immobilized sample relative to a pre-immobilization sample, immobilizing method may comprise contacting a microorganism sample with an immobilizing agent to produce a pre-immobilization sample, followed by mixing and/or dissolving the immobilizing agent throughout the pre-immobilization sample to produce a substantially homogenous immobilized sample with a viscosity that is greater than the viscosity of the microorganism sample.

Electrokinetic Immobilization

In various aspects, immobilization of microorganisms may be performed by introducing microorganisms into an immobilizing medium. In various embodiments, introducing microorganisms into an immobilizing medium may be performed using electrophoresis to electrokinetically introduce the microorganisms into the immobilizing medium. Examples of systems, devices, and reagents compatible with application of an electrical potential to a biological sample are described in U.S. Pat. Nos. 7,687,239 and 7,341,841 and U.S. application Ser. No. 14/004,145, which disclosures are incorporated by reference herein in their entirety.

In various embodiments, a microorganism sample may be contacted with an immobilizing medium and an electrical potential applied. An immobilizing medium may be a gel-immobilizing medium. The electrophoretic mobility of microorganisms in the electrical field may produce electrokinetic movement of the microorganisms from the microorganism sample into the immobilizing medium for detection and analysis. After the microorganisms have migrated into the immobilizing medium, application of the electrical field may be discontinued, leaving the microorganisms immobilized within the immobilizing medium.

In various embodiments, characteristics of the immobilizing medium and/or the microorganism sample may be adjusted to achieve various degrees of microorganism mobility into and through the gel when an electrical potential is applied. For example, an immobilizing agent and/or immobilizing agent concentration may be selected to provide a gel strength or gel pore size configured to provide a certain degree of microorganism mobility into and through a gel-immobilizing medium under certain electrophoretic conditions. Other factors, such as the size, shape, surface charge and hydrophobicity of the sample microorganisms may influence the electrokinetic potential of the microorganisms and/or be affected by electrophoretic conditions. In various embodiments, the microorganism sample composition may be adjusted or modified to manipulate a microorganism surface charge and/or hydrophobicity and adjust microorganism electrophoretic mobility. Likewise, other components of an electrophoresis system, such as the pH and/or the ionic strength of an electrophoresis buffer may influence the electrokinetic potential of sample microorganisms and be adjusted to achieve suitable electrophoretic mobility.

In various embodiments, the electrophoresis buffer and/or gel can also comprise nutrient media required for growth of the microorganisms following electrophoretic immobilization. Similarly, antibiotic agents may be added to the gel and/or buffer. Additionally, in various embodiments, certain immobilizing medium components may be added after immobilization and/or separation of the microorganisms, such as by re-equilibrating an immobilizing gel with a new buffer or medium or a medium having additional components, such as an antibiotic agent.

In various embodiments, microorganism electrophoresis into an immobilizing medium may further be used to achieve separation of sample microorganisms. For example, different types of microorganisms present in a sample may resolve to different positions of an immobilizing gel following microorganism electrophoresis based on differences in microorganism shape, size to charge ratios, hydrophobicity, or other factors that may influence the migration of a microorganism. Various non-microorganism sample components, such as other cell types from a biological specimen, sample debris, and the like, may also be present in a sample, and electrophoresis may further achieve separation of sample debris from sample microorganisms.

Various factors described above, such as the characteristics of the immobilizing medium or the electrophoretic conditions, may be manipulated to facilitate sample microorganism separation, including separation from other microorganisms and sample debris. In various embodiments, the average sample debris particle size may be substantially smaller than the average intact microorganism, and the gel strength or gel pore size may be adjusted to allow relatively rapid or unrestricted migration of a substantial portion of the sample debris, while the rate of migration of the sample microorganisms may be lower, providing for microorganism and debris separation. For example, an immobilizing agent may be selected to provide an immobilizing medium with a gel strength or gel pore size suitable for immobilization and microorganism separation for particular sample type, such as a blood culture sample, to provide optimum separation of common blood culture pathogens and sample debris. Likewise, various electrophoretic conditions, including the compositions of electrode and gel buffers, voltage, current, and run time, may be manipulated to achieve optimum microorganism separation.

Electrophoretic separation of microorganisms and debris, however, is not necessarily required, and in various embodiments, electrophoresis may be applied merely to transfer sample microorganisms into an immobilization medium simply to achieve immobilization without further achieving separation of the sample microorganisms or debris.

In various embodiments, an immobilizing medium used for electrokinetic introduction of a microorganism sample may be contained within a detection device. A microorganism sample may be contacted with the immobilizing medium by placing the microorganism in contact with the immobilizing medium in the detection device, and electrophoretic immobilization may be performed within the detection device. In various embodiments, a detection device may be a capillary tube, microcuvette, multichannel microfluidic detection device, or any other device suitable for performing electrokinetic immobilization followed by microorganism detection. For example, a sample may be introduced at an opening near the end of an elongated immobilization medium with electrodes disposed near opposite ends. Application of an electrical potential may be applied to produce electrokinetic migration of sample microorganisms (and sample debris, as applicable) into the immobilization medium. In various embodiments, the electrical potential may be applied for a time period suitable to produce migration of the microorganisms to a detection zone of the detection device. Optionally and as described above, the electrical potential may continue to be applied to produce separation of the microorganisms from one another and/or from sample debris. In various embodiments, microorganism detection and growth analysis may be performed using optical sectioning techniques.

Various other immobilizing media and electrophoresis system formats can be also used for electrophoretic immobilization methods in accordance with various embodiments. For example, an immobilization medium may comprise a horizontal agar slab gel in electrophoresis buffer in an electrophoresis chamber. The sample can be introduced to a well in the gel, followed by application of an electrical potential, with negatively charged cells (and debris particles, if present) migrating into the gel toward the positive electrode.

In various embodiments, a sample can be immobilized in a detection device, or a sample can be immobilized in an immobilizing medium located in a device other than a detection device. Samples immobilized in a device other than a detection device can be transferred to a detection device for analysis following immobilization. For example, a horizontal slab gel used for electrokinetic immobilization may be divided, such as by excising a microorganism-containing portion of the immobilizing gel medium, which can be transferred to a detection device for downstream microorganism detection and analysis.

EKC Clean Up During/Following Immobilization

In various aspects and embodiments, an electrical potential may be applied to a microorganism sample during an immobilizing process to perform separation of microorganisms and sample debris. Examples of systems, devices, and reagents compatible with application of an electrical potential to a biological sample are described in U.S. Pat. Nos. 7,687,239 and 7,341,841 and U.S. application Ser. No. 14/004,145, which disclosures are incorporated by reference herein in their entirety. An electrical potential may be applied to a pre-immobilization sample, an immobilized sample, or both. For example, in various embodiments comprising a method with an immobilizing step using a gel-immobilizing agent, an electrical potential may be applied to the pre-immobilization sample. An electrical potential can further be applied to the sample through the immobilizing or phase change step applied to the pre-immobilization sample to produce an immobilized sample. An electrical potential can still further to be applied following solidification of the gel. An electrical potential may be variously applied only to the pre-immobilization sample, only during the phase change step, only to the immobilized sample, or any possible logical combination thereof.

In various embodiments, the electrical potential may be applied with a polarity suitable to cause migration of sample elements, including microorganisms and sample debris such as microorganism cell fragments, away from a detection surface of a detection device. For example, in various embodiments in which immobilization and detection is performed using a detection device with a detection surface comprising an electrode, an electrical potential may be applied such that the electrode associated with the detection surface has a negative charge and an electrode opposite the detection surface has a positive charge. Upon application of an electrical potential, negatively charged microorganisms and debris particles may migrate away from the detection surface.

In various embodiments, the pore size or viscosity of an immobilizing medium is suitable to immobilize intact sample microorganisms following application of an electrical potential, but can permit electrophoretic mobility of intact sample microorganisms under conditions compatible with microorganism viability. In various other embodiments, the pore size or viscosity of an immobilization medium may be suitable to immobilize sample microorganisms during application of an electrical potential while permitting migration of sample debris particles.

In various embodiments, interaction of the sample elements (i.e., microorganisms and debris particles) with the (pre-immobilization or immobilized) sample medium may differentially influence a rate of migration of different sample elements. In various embodiments, factors such as the size, shape, surface area, and charge can influence the rate of migration of a sample element through an immobilizing medium. For example, sample debris such as cell fragments are generally smaller in size than intact microorganisms and may therefore migrate more rapidly than microorganisms through an immobilizing medium. In various embodiments, an immobilizing agent and/or immobilizing agent concentration may be selected to provide a gel strength or gel pore size configured to provide a certain degree of microorganism mobility into and through a gel-immobilizing medium under certain electrophoretic conditions. Likewise, other components of an electrophoresis system, such as the pH and/or the ionic strength of an electrophoresis buffer may be adjusted or modified to influence the electrokinetic potential of sample microorganisms and/or debris to achieve a desired level of microorganism separation from debris.

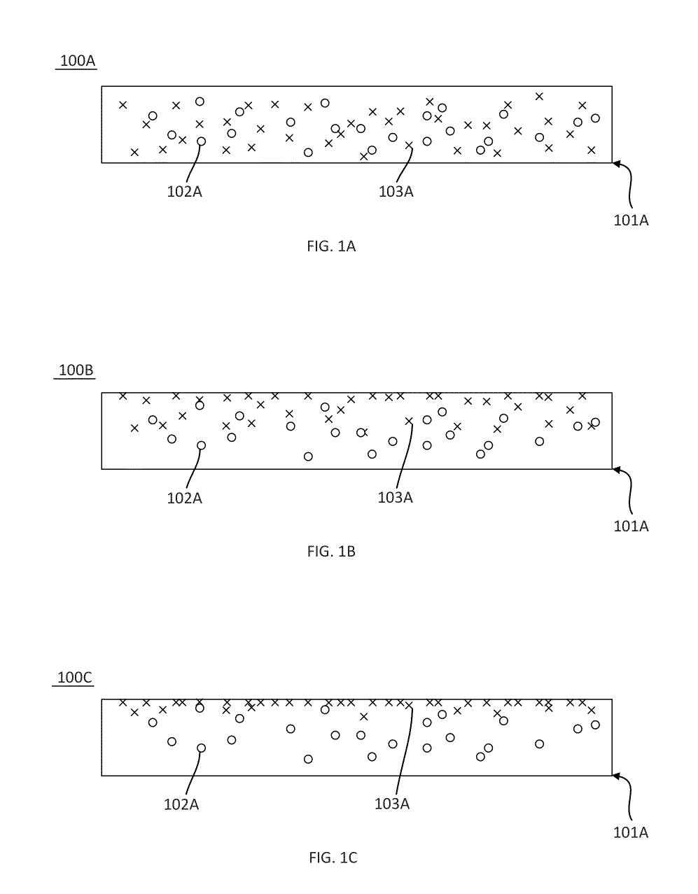

In various embodiments, an electric potential may be applied to a pre-immobilization sample or an immobilized sample to perform separation of a portion of the sample debris particles from the sample microorganisms. For example and as illustrated in FIGS. 1A-1C, in various embodiments in which immobilization and detection is performed using a detection device 100 with a detection surface 101 comprising an electrode, an electrical potential may be applied such that the electrode associated with the detection surface has a negative charge and an electrode opposite the detection surface has a positive charge. Prior to application of an electrical field, microorganisms 102A and debris particles 103A may be substantially randomly and/or uniformly dispersed throughout the pre-immobilization or immobilized sample volume of detection device 100A, as shown in FIG. 1A. Upon application of an electrical potential, negatively charged microorganisms 102 and debris particles 103 may migrate away from the detection surface, with smaller debris particles generally migrating at a greater rate than the microorganisms. After a period of time, application of an electrical potential and differential migration of microorganisms 102B and debris particles 103B may produce some separation of microorganisms from the debris particles in the sample in detection device 100B, with sample microorganisms migrating more slowly and a reduced concentration of sample debris 103B being located near detection surface 101B. A longer period of application of an electrical potential may produce a greater degree of separation of sample microorganisms 102C and debris particles 103C, as illustrated in FIG. 1C.

In various embodiments, complete separation of sample debris from sample microorganisms need not be achieved to facilitate microorganism detection. Instead, various degrees of partial separation of sample debris from sample microorganisms may be suitable to permit detection of a representative proportion of the microorganisms present in the sample. For example, in various embodiments, an electrical potential may be applied for a period of time sufficient to produce separation of approximately 5% to approximately 10% of the sample microorganisms from the sample debris. In various embodiments, an electrical potential may be applied for a period of time sufficient to produce separation of approximately 10% to approximately 20%, or approximately 20% to approximately 40%, or approximately 30% to approximately 80%, or approximately 40% to approximately 100% of the sample microorganisms from the sample debris.

In various embodiments, following electrophoretic separation of sample microorganisms from sample debris, microorganism detection is performed. In various embodiments, the electrophoretic buffer components of a pre-immobilization and/or immobilized sample composition may be compatible with microorganism viability and growth. Likewise, the conditions of the applied electrical potential, including the applied current and voltage, as well as the duration of electrical potential application, may also be compatible with microorganism viability and growth.

Sample Absorption into Immobilizing Medium

In various aspects, a method of immobilizing microorganisms can comprise contacting a microorganism sample with an absorption medium; absorbing the sample into or through the absorption medium to produce a surface-captured sample; contacting the surface-captured sample with an immobilizing medium to produce a pre-immobilization sample; and immobilizing the pre-immobilization sample.

In various embodiments, an absorption medium can comprise a medium such as a dehydrated agar gel slab or other aerogel or xerogel medium. In other embodiments, an absorption medium can comprise an absorbent filter, a filter overlaying an absorbent medium, or other solid support. The absorption medium may absorb all or a portion of the fluid component of a microorganism sample and/or cellular debris and other sub-microorganism sized particles while the sample microorganisms are excluded from the absorbent medium and remain on the surface of the absorption medium, producing a surface-captured microorganism sample. For example, in various embodiments, the pore size of an absorption medium may be suitable to absorb a microorganism sample fluid and sample debris, while being sufficiently small to exclude sample microorganism, which remain at the surface of the absorption medium. The sample may be absorbed into or through the absorption medium via passive diffusion, or the sample may be absorbed by gravity, pressure, or vacuum.

In various embodiments, a surface-captured sample is contacted with an immobilizing medium to produce a pre-immobilization sample, such as by adding a fluid (molten) immobilizing gel medium. The pre-immobilization sample is then immobilized, such as by inducing a phase change for an immobilizing agent in the pre-immobilization sample or by mixing an immobilizing agent throughout the pre-immobilization sample, as described elsewhere herein.

Confining a Microorganism to a Location

In various aspects, a method of immobilizing microorganisms comprises confining microorganisms in the immobilized sample to discrete locations in the immobilized sample volume. As defined elsewhere herein, confining a microorganism in an immobilizing medium establishes and maintains an association between the microorganism and a physical or theoretical location in the immobilizing medium over a period of time. In various embodiments, for example, a first microorganism may be confined to a first location in the immobilized sample volume, and a second microorganism may be confined to a second location in the immobilized sample volume that is distinct from the first location in response to an immobilizing step.

In various embodiments, microorganisms may be confined in association with a surface or structure of a detection device, such as a detection surface, or microorganisms may be substantially or completely surrounded with immobilizing medium. Regardless of whether microorganisms in a pre-immobilization sample are associated with a surface or are suspended within the medium, immobilizing the pre-immobilization sample in the immobilization step results in confining a first microorganism to a first location in the immobilized sample volume and confining a second microorganism to a second location in the immobilized sample volume.

In various aspects, a location to which a microorganism is confined in a sample volume may be described in terms of a volume of space. In various embodiments, a location may comprise a volume defined by a physical boundary suitable to restrict the movement of the first microorganism in the immobilized sample. The physical boundary may comprise an interface between an external surface of a microorganism and a surrounding material such as a portion of the network structure of a gel. For example, a location to which a microorganism may be confined can comprise a pore of a gel-immobilizing medium having outer boundaries defined the by structural network of the gel-immobilizing agent that the microorganism is unable to migrate or grow beyond. The volume of a location may be substantially similar to or somewhat larger than the volume of the microorganism confined to the location, or the volume may be several to many times the volume of the microorganism. A microorganism may be free to move within the location in which it is confined (i.e., within the confines of the bounded location), or the microorganism may be associated with a boundary or feature defining or contained within the location.

In various embodiments, the physical boundary defining the first location may not be a rigid or fixed boundary; instead, the boundary may be flexible and/or moveable in a manner nonetheless compatible with restriction of the movement of the first microorganism relative to the sample chamber, the detection system, or another external reference point. The material properties of the physical boundary, while suitable to restrict the movement of the microorganism in the sample chamber, may not restrict the growth of a microorganism in contact with or constrained by the physical boundary. The boundary defining the first location and/or volume of space defined by the boundary may permit the first microorganism to reproduce and generate daughter or progeny cells that may then be co-confined and co-localized with the first microorganism in the first location. In various embodiments, immobilization of a microorganism will not substantially affect a growth rate of the microorganism, for example, as compared to the growth rate of a non-immobilized microorganism. In addition, the physical boundary defining the first location may be suitable to prevent a second microorganism that is not a daughter cell or progeny of the first microorganism from entering the first location.

In various embodiments, immobilization may not be effectuated by imposition of actual physical boundaries. Rather, in various embodiments, immobilization may be described in terms of a theoretical boundary imposed, for example, by an immobilizing agent that increases the viscosity of a microorganism sample. A microorganism may be confined in an area of space that is unbounded physically (i.e., does not comprise distinct physical boundaries, such as in the case of a viscous solution or a highly porous gel having pore sizes through which a microorganism may pass) but outside of which a detected microorganism is statistically or probabilistically unlikely to travel and/or in which a detection system is able to effectively track and monitor the presence and growth of the microorganism. For example, an increase in viscosity may be suitable to reduce particle and/or microorganism movements of various types that may occur, including Brownian motion, advection, cell motility, and the like, such that a microorganism located in a defined area of space has a greater than or equal to about 75% probability of remaining in the defined area, or a greater than or equal to about 85% probability of remaining in the defined area, a greater than or equal to about 95% probability of remaining in the defined area, or any other suitable threshold probability value or range. The increase in the viscosity of the fluid may be suitable to reduce the movement of a microorganism within the immobilized sample such the microorganism is effectively confined, as the term is used herein.

In various embodiments, immobilization may be effectuated or enhanced by other mechanisms, such as by microorganism expression of surface proteins that promote agglutination and/or surface attachment. For example, coagulase-positive bacteria such as S. aureus may produce fibrin in response to the presence of prothrombin in a pre-immobilization medium. Other extracellular polymeric substances or biofilms can be produced by a microorganism and contribute to microorganism cell adherence, for example, to a surface or between cells of a clone. Likewise, still other mechanisms of microorganism attachment, such as production of pili or fimbriae, can contribute to cell attachment to surfaces or between cells and to microorganism localization. Any mechanism that contributes to confining a microorganism to an area of space within a sample volume, whether a result of an immobilizing agent provided exogenously, an agent or structure produced by a microorganism, or an interaction of the two, is within the scope of the present disclosure.

Likewise, a location of a theoretically confined microorganism may also be an area of space in which a second microorganism is statistically unlikely to enter. In various embodiments, such theoretically bounded locations may be provided by an immobilizing agent that increases the viscosity of the immobilized sample, or by a gel-immobilizing agent that may not impose a discrete physical boundary, but rather may provide an essentially predictable level of confinement of a first microorganism to a first location.

An address or physical location in an immobilizing medium may be defined in terms of two-dimensional area or three-dimensional space (i.e., a Euclidean space of two or three dimensions). A physical or theoretical address may be defined relative to a device used to support or contain the immobilized sample, such as a biosensor, detection device, or other sample holder. For example, a detection device may comprise one or more reference points or reference surfaces relative to which a first location, second location, etc., of an immobilized sample can be defined. In various other embodiments, however, a microorganism location in an immobilized sample can instead be defined with respect to another immobilized microorganism, an arbitrary or theoretical reference point, or a detection system component other than the detection device in which the immobilized sample is disposed.

The discrete physical address used to describe a location in a system can comprise any value that is meaningful with respect to the operation of a detection system, such as a Cartesian coordinate system, a cylindrical coordinate system, a spherical coordinate system, or any other suitable system. Likewise, a vector-based or coordinate-free system may also be used. Any manner of defining a location in space suitable to provide a value that may be stored in the memory of a computer-based system and used to instruct the movement of a detection device relative to a detection system by a system controller is within the scope of the present disclosure. As used herein, an address or location of a cell or other object in two or three dimensions need not be defined as a geometric point, but may also be defined as an area in a volume of space (including a volume of space with a planar orientation in systems comprising detection surface captured microorganisms (e.g., a substantially planar space)) in which a microorganism is located, such as a spherical volume of space in which the microorganism is predicted or expected to be located, or any other regular or irregular three-dimensional geometric shape that may be defined by the actual physical boundaries of the location in the immobilizing medium or that may be theoretically defined.

In various embodiments, a second location may be distinct from a first location if a detection system configured to acquire microorganism information can resolve or distinguish the different microorganisms associated with each at any time following immobilization, whether immediately following immobilization or after a first period of time following immobilization, such as a growth period. The actual physical separation of a first microorganism and a second microorganism in an immobilized sample required for the microorganisms to be distinguishable may be dependent on the detection system used, the exact nature of the sample and the presence of sample debris or other sample components. For example, sample debris can interfere with microorganism detection and resolution, thereby requiring greater actual physical resolution of the sample microorganisms for a detection system to distinguish the microorganisms. Likewise, the actual physical resolution of the first and second location may be a function of the time at which detection is performed or the time frame of an assay. For example, a first location may be distinguishable from a second location by a detection system after about 0 minutes following immobilization, or after about 10 minutes, or after about 30 minutes, or after about 60 minutes, or after about 90 minutes, or after about 120 minutes, or after about 180 minutes, or after about 240 minutes following an immobilizing step. Conversely, a first location and a second location may be distinguishable for a first period of time, but may become indistinguishable after further time has elapsed due, for example, due to microorganism growth leading to physical interference between the first microorganism and the second microorganism. Physical interference between microorganisms in a sample may occur at a physical interference rate. As used herein, a “physical interference rate” is the proportion of microorganisms in a sample for which physical interference occurs after a period of time. In various embodiments, a pre-immobilization sample microorganism concentration may be optimized to reduce a physical interference rate to beneath a target level, such as less than about 30% physical interference, or less than about 20% physical interference, or less than about 10% physical interference. For example, a pre-immobilization sample with a first pre-immobilization sample microorganism concentration may produce a 30% physical interference rate within a first growth period, while a pre-immobilization sample with a second pre-immobilization sample microorganism concentration may produce a 20% physical interference rate within the same growth period.

In various embodiments, a determination of whether a first microorganism at a first location may be distinguished from a second microorganism at a second location may be determined not by an initial ability to resolve the two microorganisms but by an assignment of an area of space to both of the first location and the second location and an assessment or prediction of the likelihood that the first location and the second location with remain distinct in the time frame and under the conditions of the assay. In various embodiments, a detection system may identify and or define a first location and assess a probability that the location will remain uniquely associated with a first microorganism (or may assign a location with a high probability of remaining uniquely associated with the first microorganism). An assessment or prediction may be based on, for example, the immobilized sample properties, which can include immobilizing medium properties such as gel strength, microorganism identity, environmental conditions of an assay, and the like. A detection system may subsequently scan or perform detection of microorganism information associated with the first location, rather than detecting and tracking the actual first microorganism. Any signal or microorganism information associated with the first location may be assumed to be associated with an attribute of the first microorganism. Likewise, as a result of the confidence in the location of a detected microorganism provided by the use of an immobilization method, any loss of signal may be interpreted as a disintegration of a microorganism, such as due to antibiotic susceptibility in the course of an AST assay, rather than due to movement or migration of the microorganism away from the location in which it was detected.

In various embodiments, an addressed location of an immobilized sample may be repeatedly visited by a detection system for acquisition of microorganism information by the detection system. A microorganism located in or associated with a discrete physical address may be repeatedly assayed or measured by a detection system in a non-destructive fashion compatible with viability and growth of a microorganism, and the detection system may acquire microorganism information useful for the determination of microorganism growth, as further described elsewhere herein.

### Immobilized Sample Format and Microorganism Distribution

Unitary and Discontinuous Immobilizing Medium Volumes

In various embodiments, an immobilizing medium may be organized in various physical formats. An immobilizing medium may have a unitary sample volume, wherein the boundaries of the immobilizing medium volume are at least partially defined by a detection device or similar container holding the immobilizing medium. An immobilizing medium may also be discontinuous, wherein the immobilizing medium comprises physical boundaries defining sub-volumes of an immobilized sample. Various physical formats of an immobilizing medium are described in greater detail below.

In various embodiments, the immobilized sample volume may be a unitary volume. A unitary sample volume can comprise a substantially continuous, integrated immobilizing agent network throughout the sample volume. For example, an immobilized sample contained within a chamber of a detection device may comprise an immobilizing agent network that extends substantially uninterrupted or without an intervening boundary throughout the sample volume and/or the chamber volume. The fluid phase of a unitary gel-immobilized sample may be in fluid communication throughout the volume of a biosensor sample chamber. Stated differently, all solid-phase bounded domains contained within the immobilized sample may be in fluid communication with one another, and the physical network of the immobilizing agent may likewise extend continuously and un-interrupted throughout the volume of a detection device chamber. However, the fluid phase of a unitary gel-immobilized sample need not be in fluid communication throughout all solid-phase bounded domains in the immobilized sample, and all or a portion of the bounded domains may be in fluid isolation from other domains (i.e., not in fluid communication).

In various aspects, an immobilized sample may be discontinuous. A discontinuous immobilized sample can comprise an overall sample volume that further comprises a plurality of sub-volumes that are separated from one another by a boundary that partially or wholly interrupts the immobilizing agent network between sub-volumes of an immobilized sample. For example, a discontinuous immobilized sample may comprise a plurality of microdroplets. Each microdroplet may comprise a small volume of an immobilizing medium defined by a boundary.

In various embodiments, an immobilizing medium in a microdroplet format can comprise a liquid or a gel. The boundary can be an interface with a surrounding material, such as an interface between an aqueous liquid microdroplet and a surrounding non-aqueous fluid (i.e., an emulsion), or the boundary may be a membrane, shell, or other permeability barrier. The boundary may be suitable to confine a microorganism within a microdroplet. The boundary may be suitable for exchanging other non-microorganism objects, such as small molecules, ions, macromolecules, and the like, or the boundary may be impermeable or selectively permeable to various non-microorganism objects. A microdroplet may be approximately spherical, or a microdroplet may have any other suitable shape. Various formats and properties of microdroplets and methods for producing the same are described, for example, in U.S. Pat. No. 4,959,301, which disclosure is incorporated by reference herein in its entirety.

Detection Devices and Sample Chambers

In various embodiments, an immobilized sample volume may be defined or partially defined by a detection device the immobilized sample is contained by or confined within. A detection device can include devices such as biosensors, microfluidic detection devices, microfluidic cartridges, and other specialized devices may be used to facilitate microorganism immobilization and detection. Examples of devices, systems, and methods that enable individuation, immobilization and detection of discrete microorganisms, microorganism identification and AST testing in accordance with various embodiments of the present disclosure are described in detail in U.S. Pat. Nos. 7,341,841 and 7,687,239 and International Patent Application No. PCT/US2014/0030745, which disclosures are herein incorporated by reference in their entirety. Any type of container or device suitable to hold an immobilized sample for detection and analysis by a detection system is within the scope of the present disclosure. For example, in various embodiments, a pre-immobilization sample may be pumped or injected into a biosensor, such as a device comprising one or more microchannel flowcells. In various other embodiments, an immobilized sample may be contacted with or introduced to a detection device after immobilization, such an immobilized microorganism sample excised from larger immobilizing medium.

In various embodiments, a pre-immobilization sample may be divided into a plurality of separate immobilized samples from each pre-immobilization sample. For example, a pre-immobilization sample may be pumped or injected into a biosensor device comprising a plurality of parallel microchannel detection chambers (also referred to simply as “channels”) prior to immobilization. Each separate parallel channel may be independent or isolated from each of the other channels following introduction of the pre-immobilization sample and/or immobilization. Likewise, the immobilized sample of each channel may be considered a separate immobilized sample for purposes of the present disclosure.

In various embodiments, each of the plurality channels comprising an immobilized sample may be placed in a condition. In various embodiments, each channel/immobilized sample condition may be different from and independent of the condition of the other channels/immobilized samples. For example, an antibiotic agent may be added to various channels at different concentrations for AST testing and MIC determination.

In various embodiments, an immobilized sample may comprise a microvolume-immobilized sample. For example, in various embodiments, a microvolume sample may be less than about 5000 μl, or less than about 2000 μl, or less than about 1000 μl, or less than about 500 μl, or less than about 100 μl, or less than about 50 μl.

In various embodiments, the microvolume format and/or the short duration in which growth analysis is performed may facilitate detection of growth of aerobic organisms in an immobilizing medium. For example, enumeration or detection of growth of obligate aerobic microorganisms may not be feasible with traditional pour plate methods using petri dishes and macroscopic, end-point detection of colonies. Clones may be unable to grow sufficiently in a large format solidified gel medium due to inadequate oxygen permeability and/or oxygen starvation prior to growth to macroscopically detectable colony size, resulting in false negative results. In various embodiments, a microvolume immobilizing medium format may comprise a suitable surface area to volume ratio to afford sufficient oxygenation of the medium for aerobic organism growth. Similarly, turbulent pre-immobilization medium flow during sample preparation, mixing, and/or introduction into a biosensor flowcell channel may effectively aerate the immobilizing medium. In various embodiments, the reduced time frame required for microscopic detection and growth determination may reduce the potential for oxygen starvation-related growth arrest in an immobilizing medium in a time frame in which detection of growth is performed.

In various embodiments, a microvolume immobilizing medium format may be suitable for detection of anaerobic microorganisms. A microvolume immobilizing medium may be prepared using techniques to minimize aeration or oxygenation of the medium. A microvolume immobilizing medium may comprise oxygen scavengers such as thioglycolate, pyruvate, L-cysteine hydrochloride, catalase, peroxidase, oxyrase, and the like, to produce an immobilizing medium suitable for microaerophilic microorganisms or anaerobic microorganisms.

### Microorganism Distribution

2D Microorganism Distribution

In various aspects and embodiments, a surface-capture step may be performed as an initial step prior to immobilization. The volume of a sample to be analyzed directly influences the effort required to detect and track microorganisms in a sample, with relatively large sample volumes placing increased demands on an analytical system with respect to data acquisition and processing. Surface capture of microorganisms in a sample can be performed to reduce the effective sample volume that must be analyzed by driving cells suspended in bulk solution (i.e., a planktonic state) to a surface bound (sessile) state on a capture surface or a detection surface. Surface capture can facilitate detection and tracking by concentrating and/or individuating the microorganisms in a sample to a known region of the sample volume.

Capture of Microorganisms

In various embodiments, a microorganism detection system comprises a computer-based system and may be a bench top instrument that combines a disposable microfluidic cartridge with automated microscopy and image analysis software. The detection system can include, among other features, automated sample distribution to multiple on-board analysis chambers providing integrated electrokinetic concentration and imaging, electrophoretic concentration to a capture and imaging surface using transparent indium tin oxide (“ITO”) electrodes and redox buffer system, phase contrast, darkfield, fluorescence, and confocal microscopy, XYZ motion control including autofocus, off-board (instrument-based) pumps and fluid media, on-board reagent reservoirs (antibodies, stains, antibiotics), and active on-device valving for fluidic network control.

Evaluations can be performed using a computer-based microorganism detection system, which in various embodiments may be a bench top instrument that combines a disposable microfluidic cartridge with automated microscopy and image analysis software. The detection system can include, among other features, automated sample distribution to multiple on-board analysis chambers providing integrated electrokinetic concentration and imaging, electrophoretic concentration to a capture and imaging surface using transparent ITO electrodes and redox buffer system, phase contrast, darkfield, fluorescence, and confocal microscopy, XYZ motion control including autofocus, off-board (instrument-based) pumps and fluid media, on-board reagent reservoirs (antibodies, stains, antibiotics), and active on-device valving for fluidic network control with off-board specimen preparation (i.e., simple centrifugation or filtration sample preparation). The detection system can provide rapid concentration of microorganisms to a capture surface and a detection surface using electrokinetic concentration. In various embodiments, a detection surface may not be associated with a capture surface or microorganism capture. Targeted microorganism identification can be performed by fluidic introduction of species-specific antibodies followed by fluorescently labeled secondary antibodies, with automated epi-fluorescent microscopy. In various embodiments, individual clones can be mapped, and growth rate determination exploits registered time-lapse image analysis, processed to derive growth rate information (e.g., doubling times and growth rate constants) using the detection system. The detection system can also provide on-board, near real-time antibiotic susceptibility testing (AST).

In various embodiments, a detection device can comprise a flowcell for use with a microorganism detection system. The flowcell can include ITO coated glass as top and bottom layers, optionally with an adsorptive chemical coating on the bottom surface (i.e., the capture and/or detection surface). Intermediate structure sandwiched between the top and bottom layers may form one or more sample chambers within the detection device. A sample containing microorganisms may be introduced to the detection device and/or each sample chamber and a potential applied. Since bacteria are generally negatively charged, they migrate to the positively charged surface, where they may adsorb to the chemical coating forming the capture and detection surface.

The capture surface may facilitate localization of microorganisms in a sample at or near the detection surface. In various embodiments, localization of microorganisms at or near a detection surface facilitates microorganism detections. A variety of methods may be used for the capture of microorganisms onto surfaces in accordance with aspects embodying the invention. In general, these fall into two categories: specific and non-specific capture. “Capture” in this context means that the microorganisms are associated with the detection surface(s) such that they do not significantly move or detach under conditions of a given assay. For example, this association is generally strong enough to allow washing steps without removing the microorganisms from the surface. In general, capture relies on non-covalent forces such as electrostatic interactions, hydrogen bonding, hydrophobicity, etc., although in some instances, covalent attachment (including for example cross-linking) can be employed. Activated cross-linking may be achieved, for example, via thermal or light induced means.

In general, there are a variety of techniques, including state of the art techniques, which can be used to non-specifically capture microorganisms onto detection surface(s). As above, these techniques generally rely on hydrogen bonding, electrostatic and hydrophobic interactions, which can be used either singly or in combination.

There are a number of known materials that are “sticky” to either or both of microorganisms and/or biological molecules. These include any number of biological molecules and polymers, including, but not limited to, poly-ionic surfaces, particularly poly-cationic surfaces when the microorganisms have an overall negative charge, including polyamino acids (e.g. polylysine), and fibronectin. Furthermore, it is well known in the art that species of bacteria bind selectively to certain molecules. For example, it is well known that Escherichia coli binds mannose surfaces selectively. Streptococcus and Staphylococcus organisms bind the Fc portion of antibodies via protein A mechanism. These receptor ligands may be utilized to immobilize bacteria on surfaces highly hydrophobic surfaces, such as polystyrene, are generally “sticky” to, microorganism and can also be used.

One polymeric surface of interest is OptiChem, as described in U.S. Patent Publication No. 2003/0022216, which is a member of a class of “hydrogel” surfaces (including also CodeLink by Amersham) that are highly porous and which generally support, because of this porosity, the diffusion of redox mediators and interactions with the electrodes needed for the electrophoresis of microorganisms. This can be modified with particular groups to enhance non-specific adhesion, including diethylenetriamine (useful to enhance electrostatic interactions), and Tris and ethanol amine (useful to enhance hydrogen bonding). It can also be modified with hydrophobic moieties, which can include benzenes, naphthalenes, and compounds containing such moieties, which are preferably substituted with amines or sulfhydryls so that they can be conveniently linked to hydrogels.

Spacing

In one aspect of the invention, the spacing of microorganisms on the surface of interest is controlled. Bacteria electrophoretically transported from bulk solution to a surface tend to form semiorganized clusters on the surfaces, due to electrohydrodynamic flow. For QM purposes, a majority of the cells should be associated with the surface at individual discrete sites, that is, clustering is limited. There are a variety of ways to accomplish this. In one aspect, the viscosity of the electrophoretic solution is increased by adding a viscosity agent. Suitable viscosity agents include glycerol, saccharides, and polysaccharides such as dextrans, and polymers such as polyethylene glycol. These agents can be added at different concentrations, depending on their viscosity; for example, 10-25% glycerol, with 20% being a particular aspect, is useful. In some cases, other reagents may be added to reduce this “clustering” effect, optionally in conjunction with viscosity agents and the techniques outlined below. For example, surfactants, proteins such as albumins, caseins, etc., specific inhibitors of cellular adhesion, polymeric materials such as polyethylene glycol, and dextran can be added to reduce the clustering.

In another aspect, fluidic design and electrokinetic electrode geometry may be advantageously employed to provide or augment the spacing of the microorganisms on the surface.

In yet another aspect, the spacing of the microorganisms on the surface is accomplished by controlling the density of either specific capture ligands or components that contribute to non-specific binding on the detection surface. For example, when specific capture ligands are used, the concentration of the ligand on the surface is controlled to allow a spatial density that allows the binding of individual microorganisms at discrete sites that are spatially separated. In one aspect, the separation distance is greater than the diameter of several microorganisms, such that a single microorganism bound at a discrete site can undergo several cycles of cell division and still be detectably distinct from other microorganisms bound at neighboring regions. The density of the capture ligands will depend in part on the size of the microorganism to be evaluated, as well as the concentration of the microorganism in the sample. With respect to concentration, the number of microorganisms added to the system for binding to the capture surface can be regulated. In general, the number of microorganisms should be balanced with the size of the capture surface such that the center-to-center distance between the microorganisms has as a median at least 10 microns, and more preferably 20 microns, and even more preferably 40 microns. This distance will ensure that even after a number of divisions, wherein the sibling microorganisms from a single founder will number 16 or 32, most minicolonies (also referred to as “clones”) will remain distinct and not overlapping.

In various embodiments, washing steps may be performed following microorganisms capture on a detection surface to provide new nutrients for growth conditions. That is, there may be one buffer system for use in the electrokinetic concentration step which is exchanged after microorganism capture at the detection surface. Alternatively, and as described in greater detail in the following section, the buffer system used in the electrokinetic concentration step may be exchanged for an immobilizing medium that further facilitates microorganism detection and various assays that may be performed.

However, surface capture alone is insufficient to ensure that a clone can be tracked over a growth period. Many bacterial species tend to replicate and propagate in a biofilm mode of growth. During biofilm formation, some progeny cells may transition from sessile to planktonic states, resulting in dispersal of the cells. A transition to a planktonic state can be driven through a passive or an active process. Examples of passive processes include spurious flow and diffusion processes, and examples of active processes include chemotaxis and motility, such as swarming and swimming. During analysis of a surface-captured or sessile clone's response to a stimulus, any loss of microorganism cells due to a transition to a planktonic state can confound the process of assigning cells to founder clones or assessing growth of an identified clone associated with a capture or detection surface. If the assignment process is confounded to the point of confusion, the ability to determine a clone's response to stimulus is lost and the ability to differentiate between growth arrest or lysis of member cells and cell loss due to a transition to a planktonic state is lost.

The transition of progeny cells to a planktonic state can occur within about an hour of growth of a founder cell, whereas antimicrobial effects may require more than an hour of characterization to determine susceptibility.

In the absence of immobilization, proper tracking of sessile cells requires high frequency time interval monitoring of clone growth in order to properly assign progeny cells to founder clones. Non-immobilized sessile bacteria may be motile, and progeny cells may be capable of swimming or drifting multiple cell diameters away from the founder cells. The velocity, magnitude, and direction movement of progeny cells relative to the founder cell (due to drift or bacteria motility) is not generally known a priori. As such, the assignment of cells to clones requires a sufficient time lapse imaging frequency such that the distance of progeny cells from the founder cells is a fraction of the bacterial cell size in order to maintain integrity of the progeny cell assignment to the founder cells (i.e., permits a tracking function to be performed). Motile bacteria can sustain velocities up to a hundred microns per second, meaning that the frequency of time lapse imaging frequency must be on the order once every few seconds in order for tracking to have the potential to be successful. Likewise, as described above, the volume of the sample to be analyzed also directly affects the analytical effort required for tracking, with larger volumes placing increased demands on the system with respect to performing accurate microorganism tracking.

Immobilization of founder cells confines progeny cells, preventing motile or planktonic microorganisms from moving or migrating in a manner that interferes with accurate tracking of clone growth for extended time periods and accommodating detection system sampling frequencies with up to orders of magnitude lower time. This means lower image acquisition and data processing demands and enables a detector to move away from a first location to a second location and to return back to that first and second location, resulting in the ability to analyze more area and improving analytical sensitivity. For various detection technologies, surface-capture of sample microorganisms, such as on a detection surface, is not necessarily required. Instead, in various methods and systems, and initial capture step can be performed simultaneously with immobilization in a single step, such as, for example, when the cells of a sample are entombed in agar.

In various embodiments, a sample comprising one or more microorganisms may be introduced to a detection device comprising a chamber and a detection surface. The microorganisms in a sample may be captured on the detection surface prior to immobilization and detection of the microorganisms. For example, a sample comprising organisms may be introduced to a detection device comprising a microfluidic detection device suitable for performing EKC, as described in U.S. Pat. Nos. 7,687,239 and 7,341,841. The microorganism may be suspended in and introduced to the detection device in a buffer compatible with the EKC process. Following introduction of the sample comprising the microorganisms, an electrical potential may be applied to the sample using the detection device, wherein application of the electrical potential results in migration of the microorganisms toward a detection surface that may be treated with a microorganism capture film or surface treatment suitable to maintain the microorganisms in association with the capture surface, with each microorganism being associated with a discrete location on the capture surface. Following capture of the microorganisms in the sample on the capture surface, the sample buffer used for introduction of the sample into the detection device and EKC may be exchanged with an immobilizing medium.

In various other embodiments, microorganisms may be embedded in an immobilizing medium, followed by contacting the surface of the immobilizing medium containing the immobilized microorganisms with a detection surface. Microorganisms embedded in a surface of a gel medium may be immobilized as a result of or following embedding in the gel medium, and a portion of the gel medium comprising the gel surface with the embedded sample microorganisms may be contacted with a detection surface of a detection device, followed by microorganism detection as further described below.

In various embodiments, the immobilizing medium may comprise Mueller-Hinton agar (“MHA”) or similar agar- or agarose-containing microbiological medium. The MHA may be introduced to the detection device at a temperature (i.e., the composition temperature) at which the MHA is molten and/or substantially flowable into and through the detection device chamber. In various embodiments, the temperature of the MHA or other immobilizing medium is sufficiently high that the medium is molten and flowable, but not so high as to result in non-viability of the microorganisms captured on the capture surface of the detection device and exposed to the molten medium. In various embodiments, for example, for MHA comprising Noble agar (e.g., Sigma-Aldrich A5431; Sigma-Aldrich, St. Louis, Mo.) with a gelling temperature of 32-39° C., the MHA is introduced to the detection device when the temperature of the MHA is from about 39° C. to about 44° C., or from about 39° C. to about 42° C., or from about 39° C. to about 41° C., or from about 39.5° C. to about 40.5° C. In an embodiment, MHA is introduced to the detection device when the temperature of the MHA is about 40° C. In various embodiments, other gel-immobilizing agents with different gelling temperatures may be selected, for example, based on the temperature requirements of a target microorganism. For example, a low melting point agarose may be used as a gelling agent. In various embodiments, for example, for MHA comprising low melting point agarose (e.g., UltraPure Low Melting Point Agarose, Life Technologies, NY, USA), the MHA is introduced to the detection device when the temperature of the MHA is from about 25° C. to about 39° C., or from about 25° C. to about 37° C., or from about 25° C. to about 35° C., or from about 25° C. to about 32° C., or from about 25° C. to about 29° C. In an embodiment, MHA is introduced to the detection device when the temperature of the MHA is about 27° C. A variety of agars and agaroses with different material properties are commercially available and may be selected for use in an immobilizing medium. Following introduction of the MHA, the MHA is cooled to induce a phase change in the immobilizing agent and immobilize the sample microorganisms. The agar or agarose solidifies, immobilizing the captured cells in association with the capture surface. In various embodiments, the presence of the MHA in the detection device substantially prevents microorganism movement away from the detection surface in the detection device and/or migration of a captured microorganism located at a first physical location on the detection surface to a second location on the detection surface. Detection of the microorganisms may proceed before or after the MHA or other immobilizing medium has solidified.

Immobilization of a first microorganism at a first location on a detection surface using an immobilizing medium such as, for example, MHA, can permit physical growth or expansion of the first microorganism. Likewise, immobilization of the first microorganism on the detection surface can permit growth of the first microorganism by reproduction and production of progeny cells. In various embodiments, progeny cells will generally be immobilized and co-localized at or near the detection surface in approximately the same plane as the first microorganism progenitor. In various embodiments, the physical interface between the detection surface or other surface of a detection device and the immobilizing medium may provide the least restrictive paths of physical expansion. In various embodiments, growth of the immobilized microorganisms may occur substantially in a planar or two-dimensional orientation along the detection surface (i.e., in the x-axis and y-axis directions). However, some growth may occur in a direction away from the plane of the detection surface into three-dimensional space (beyond the inherent three-dimensional space necessarily present due to the height of the first microorganism; i.e., in the z-axis direction). Progeny cells produced in a direction extending substantially orthogonal to the detection surface will likewise be immobilized by the immobilizing medium and co-localized with the first microorganism. Clone growth located out of contact with the detection surface (i.e., growth into the immobilizing medium in the z-axis direction) may be detected by a detection system in accordance with various embodiments of the present disclosure.

3D Microorganism Distribution

In various embodiments, microorganisms for detection with a detection system need not be captured on a surface or associated with a detection surface prior to immobilization. In various embodiments, immobilized microorganisms may be suspended in the volume of a pre-immobilization sample prior to performing an immobilizing step, producing an immobilized sample having sample microorganisms distributed in three dimensions throughout the immobilized sample volume. For example, microorganisms in a pre-immobilization sample may be introduced into or contacted with a detection device and immobilized in a three-dimensional space. The three-dimensional space may comprise, for example, all or a portion of the volume of a detection device.

As used herein, the concept of microorganisms “suspended” in a medium includes, for example, planktonic microorganism cells. Following an immobilizing step, however, suspended or planktonic cells are no longer “free-floating,” but are instead confined to a discrete location within the immobilized sample volume, as described in greater detail herein. Suspended cells in an immobilized sample volume may be substantially surrounded by the immobilizing medium, although some cells located near boundaries of the detection device may be in contact with and partially confined by a surface of the detection device.

A three-dimensional space of a detection device may have any shape or configuration suitable to accommodate various optical and non-optical microorganism detection systems and methods, including traditional devices or sample holders that may be compatible with various detection systems, such as slides, petri dishes, chambers, multiwell plates, cuvettes, test tubes, microfuge tubes, capillary tubes, microfluidic detection devices, and the like. In various embodiments, custom detection devices with custom sample chamber configurations are possible. The volume of the sample chamber may vary and be dependent on the detection system used to obtain microorganism information and whether the microorganisms are immobilized in association with a detection surface in two dimensions or are immobilized dispersed in three dimensions in the sample chamber. Likewise, the size, number, and configuration of one or more detection surfaces of a sample chamber or detection device may vary. A detection surface may be any surface of a sample chamber or detection device that is suitable for or compatible with acquisition microorganism information for microorganisms in a sample chamber of a detection device. Any detection device having any sample chamber configuration suitable for microorganism detection may be used.

In various embodiments, a sample comprising microorganisms already immobilized in an immobilizing medium is contacted with, or introduced to, a detection device. In other embodiments, a sample comprising microorganisms to be detected is introduced to a sample chamber of a detection device followed by immobilization of the microorganisms in the sample chamber. For example, a sample comprising microorganisms may be added to a liquid gel immobilizing medium such as molten MHA, followed by introduction of the medium containing the microorganisms into a detection device and solidification of the immobilizing medium in the detection device. In various embodiments, a sample comprising microorganisms is combined with a polymer that may be chemically cross-linked to form an immobilizing gel medium after introduction of the sample to the detection device.

### Detection Systems and Methods of Acquiring Microorganism Information

In various aspects, a microorganism detection system is used to detect immobilized microorganisms in a detection device and to acquire information regarding the immobilized microorganisms. Various systems and methods for acquiring microorganism information are described herein. Examples of devices, systems, and methods that enable detection and acquisition of microorganism information in accordance with various embodiments of the present disclosure are described in detail in U.S. Pat. Nos. 7,341,841 and 7,687,239 and International Patent Application No. PCT/US2014/0030745, which disclosures are herein incorporated by reference in their entirety.

In various aspects, acquisition of microorganism information is performed for individual immobilized microorganisms (i.e., a cell or a clone derived from a single CFU). The acquired microorganism information may be used to identify and characterize one or more immobilized microorganisms in a specimen or sample and/or determine growth of individual immobilized microorganisms over a period of time, rather than assessing growth at a bulk population level. For example, a microorganism sample analysis can comprise viable microorganism analysis and antimicrobial agent susceptibility testing for individual immobilized microorganisms in a sample.

Detection of growth may be performed within a short period of time following immobilization, and the ability to analyze or measure changes in the attributes of immobilized microorganisms facilitates detection of growth in a short time frame in comparison to traditional microbiological methods, such as minutes or hours rather than days. For example, growth can be detected in less than the amount of time necessary for the observation of clones with the naked eye (i.e., formation of visible colonies). In various embodiments, detection of growth may be performed in less than about 12 hours, or less than about 8 hours, or less than about 6 hours, or less than about 4 hours, or less than about 3 hours, or less than about 2 hours, or less than about 1 hour, or less than about 30 minutes.

Similarly, growth may be detected within a time frame of only a few, several (i.e., 4-9), or tens of cell doubling events of a microorganism, rather than the hundreds or thousands of doubling events that may be required to assess growth and/or susceptibility with traditional methods. In various embodiments, analysis of growth can be performed within a time frame within which a microorganism present in the sample can undergo from 1 to about 10 doubling events, with from about 1 to about 4 being particularly useful, and 1 to 2 being ideal in situations where the “time to answer” is being minimized. In various embodiments, analysis of growth can be performed in a time frame within which a microorganism present in the sample undergoes less than about 100 doubling events, or less than about 50 doubling events, or less than about 20 doubling events, or less than about 10 doubling events, or less than about 7 doubling events, or less than about 5 doubling events, or less than about 4 doubling events. In various embodiments, analysis of a microorganism sample, including viable microorganism analysis and antimicrobial agent susceptibility testing, does not require an initial growth of microorganisms (either liquid or solid) prior to an evaluation of growth; rather, direct-from-specimen biological samples may be analyzed with no growth or culturing prior to the assay.

In various aspects, “detecting growth” may be performed using a computer-based system, configured to integrate microorganism information associated with the detection and/or measurement of one or more attributes of a microorganism over a period of time. In various embodiments, a method may comprise: detecting a microorganism, acquiring first microorganism information by a microorganism detection system at a first time; acquiring first microorganism information by a microorganism detection system at a second time; and detecting growth of the first microorganism based on a change in microorganism information from the first time to the second time. In various embodiments, systems and/or methods of microorganism detection may provide real-time or near real-time acquisition of microorganism information, and the difference in time between a first time and a second time at which microorganism information is acquired can be very small, for example, from about 10 minutes to about 30 minutes, or from about 5 minutes to about 15 minutes, or from about 1 minute to about 5 minutes, or from about 30 seconds to about 2 minutes, or from about 5 second to about 1 minute, or from about 1 second to about 30 seconds. Detection of growth may be based on evaluation of microorganism information from a plurality of time points, such as about 50 to about 100 time points, or from about 10 to about 50 time points, or from about 5 to about 20 time points, or from about 2 to about 10 time points, or from about 2 to about 5 time points.

Detection of growth and/or a determination of a growth rate, or a lack thereof, for a microorganism need not be based solely on a direct or absolute assessment of cell viability, change in size or mass, performance of metabolic processes (i.e., homeostasis, anabolic, or catabolic processes), reproduction, or the like, but instead may be based on a probabilistic assessment that a measured change in one or more attributes is likely to correspond to growth. Thus, in various embodiments, detection of growth and/or determination of a growth rate may be performed based on measurement of a change in one or more attributes over time and a determination of a statistical probability of whether the measured change corresponds to growth, as compared to a control or reference.

Once the microorganisms present in the sample have been immobilized, individual microorganisms can be interrogated (e.g., optically, spectroscopically, bioelectroanalytically, etc.) using the microorganism detection system to measure an attribute of, characterize, and/or identify the microorganisms in the sample. The interrogation or detection of an attribute of a microorganism can take place in any suitable manner, including non-invasive techniques that do not interfere with the integrity or viability of the microorganism. Expressed differently, attributes of a microorganism present in a sample can be detected and measured while the microorganism remains in a detection device and/or remains intact. Moreover, in various embodiments, attributes of a microorganism may be detected while the organism remains viable and/or capable of undergoing growth. An attribute of a microorganism may include an intrinsic property of the microorganism, such as a property of the microorganism present in the absence of any additional, exogenously provided agent, such as a stain, dye, binding agent, or the like. An attribute of a microorganism can also include a property that can only be detected with the aid of an exogenously added agent that may facilitate detection of the microorganism, directly (such as by staining the microorganism) or indirectly (such as by reacting with a secreted metabolite). The ability to identify the microorganisms in a non-invasive manner, optionally coupled with keeping the sample contained (e.g., sealed within a detection device or biosensor) throughout the analysis process, along with automation of the procedure, may contribute to reduced handling of potentially pathogenic samples and may increase the safety of an identification or AST process relative to traditional clinical microbiological methods. Furthermore, the ability to characterize and/or identify microorganisms, for example, by direct interrogation of a direct-from-specimen sample without further processing of the sample (e.g., cleanup, concentration, dilution, centrifugation and resuspension, plating, or pre-growth of colonies, etc.) can greatly increase the rapidity with which identification/characterization can be made.

Any of a number of detection systems and/or methods that may provide an ability to detect an attribute of a microorganism may be used in accordance with various aspects and embodiments. These include detection systems using methods such as brightfield imaging, darkfield imaging, phase contrast imaging, fluorescence imaging, upconverting phosphor imaging, chemiluminescence imaging, evanescent imaging, near infra-red detection, confocal microscopy in conjunction with scattering, surface plasmon resonance (“SPR”), atomic force microscopy, and the like. Likewise, various combinations of detection systems and/or methods may be used in parallel or in complementary fashion to detect one or more attributes of a microorganism in accordance with the present disclosure.

In various embodiments, a computer-based detection system may detect, measure, track, and analyze individual immobilized microorganisms based on optical image data, such as digital photomicrographs acquired using any of a variety of methods and imaging modes well known to a person of skill in the art, various examples of which are further described below. An optical detection system may measure microorganism attributes and perform data analysis using measured signal intensity values, such as, for example, pixel intensity values from a digital image. Systems and methods of microorganism detection are described in U.S. Pat. Nos. 7,687,239 and 7,341,841 and International Patent Application No. PCT/US2014/0030745, which disclosures are incorporated by reference herein in their entirety.

In various aspects, acquisition of microorganism information may be performed using optical detection of microorganisms in a plurality of focal planes through a sample comprising microorganisms immobilized in a three dimensional space (i.e., the microorganisms are not concentrated at a detection surface and/or capture surface). The image data acquired in each focal plane is referred to herein as an “optical cross section” or “optical section” of the immobilized sample. The image data may be acquired by the detection system through a detection surface of the detection device. In various embodiments, the focal plane may be coplanar with a detection surface (i.e., the direction of movement for acquisition of successive optical cross sections is orthogonal to the detection surface), or the focal plane may be angled with respect to the detection surface (i.e., the direction of movement for acquisition of successive optical cross sections is non-orthogonal to the detection surface). In various embodiments, an optical cross section comprises at least one image through a cross section of the sample volume. The image acquired for a cross section of the sample may comprise the entire physical cross section of a sample chamber, or it may comprise a portion of the physical cross section of the sample chamber. Multiple optical cross sections that are fractions of the physical cross section of a sample chamber may be integrated or assembled to create a composite optical cross section of a sample chamber.

Acquisition of microorganism information for a three dimensional sample may comprise obtaining at least two optical sections. An objective position of the detection system may be changed with respect to a first microorganism position in the sample volume in at least one of an x-axis direction, a y-axis direction, and a z-axis direction. In various embodiments, the objective position may be changed with respect to the first microorganism position in the z-axis direction, with the detection system determining a first microorganism focal plane objective position producing an optimum first microorganism focus condition. The detection system can acquire first microorganism information at a first time point. The objective position may be changed to a second focal plane objective position and returned to the first microorganism focal plane objective position to acquire first microorganism information at a second time point. Microorganism information may be acquired for two or more time points for a detected microorganism from a time-lapse series of images taken at the first microorganism focal plane objective position.

In various embodiments, an objective aperture may be changed between a first numerical aperture and a second numerical aperture. The first numerical aperture may be used to determine a first microorganism preliminary focal plane objective position, and the second numerical aperture can be used to determine the first microorganism focal plane objective position. In various embodiments, a second microorganism preliminary focal plane objective position is determined prior to determining the first microorganism focal plane objective position.

In various embodiments, acquisition of microorganism information may comprise tens, hundreds, or thousands of optical cross sections of a sample chamber.

In various embodiments, a motion control unit may be controlled to move the detection device relative to the detection system in small steps while acquiring the optical cross sections. The size of the steps may be below about 100 micrometers, or below about 50 micrometers, or below about 10 micrometers, or below about 1 micrometer, or below about 0.1 micrometers. In various embodiments, a component of the detection system may be moved relative to the detection device. Either the detection device, the detection system, or both may be moved by a motion control unit of a system to acquire optical cross sections of a sample.

In various embodiments, the size of a step may be varied from step to step. The size of the steps may be determined to be equal to depth of field (DOF) of the detection system or a fraction thereof, or it may be equal to a distance that is a multiple of the DOF. The size of the step may be determined by information acquired from an image. For example, if an object is located in an optical cross section, the size of the next step could be determined based on the DOF. On the other hand, if no object is located in an optical cross section, the size of the step may be adjusted to maximize the search efficiency and minimize the time required for detection of a maximum number of microorganisms in a detection device chamber.

In various embodiments, at least one of an illumination wavelength and an illumination intensity may be adjusted in response to a sample parameter to compensate for at least one of a sample light scattering and a sample light absorption. A sample parameter may be dynamically determined or predetermined, and can include, for example, a debris particle concentration, a microorganism concentration, an immobilizing agent composition, an immobilizing medium thickness, sample type or source, and the like.

In various aspects, microorganism information acquired by the detection system is processed by the detection system to detect growth of a microorganism in the sample. Detection of growth may be performed in accordance with the methods and systems described in U.S. Pat. Nos. 7,687,239 and 7,341,841 and International Patent Application No. PCT/US2014/0030745, which disclosures are incorporated by reference herein in their entirety. In various embodiments, and image registration shift is performed between sequential images in a time-lapse series. The registration shift may be performed by a translation of image data in one of a two-dimensional plane or a three-dimensional space.

In various embodiments, spectroscopic methods can be used to detect one or more attributes of the microorganisms. These may include intrinsic properties, such as a property present within the microorganism in the absence of additional, exogenously provided agents, such as stains, dyes, binding agents, etc. Optical spectroscopic methods can be used to analyze one or more extrinsic attributes of a microorganism, for example, a property that can only be detected with the aid of additional agents. A variety of types of spectroscopy can be used, including, for example, fluorescence spectroscopy, diffuse reflectance spectroscopy, infrared spectroscopy, terahertz spectroscopy, transmission and absorbance spectroscopy, Raman spectroscopy, including Surface Enhanced Raman Spectroscopy (“SERS”), Spatially Offset Raman spectroscopy (“SORS”), transmission Raman spectroscopy, and/or resonance Raman spectroscopy or any combination thereof.

Non-optical methods may also be used for detection, data acquisition, and analysis, and any form of quantitative data or measured signal intensity values that may be acquired by any of a variety of measurement systems may be suitable for analysis by the detection system. In various embodiments, microorganism information acquired by a non-optical method may be processed in a manner similar to that for pixel intensity values derived from image data.

In various aspects and embodiments, a system and methods are provided to identify individuated microorganisms and evaluate microorganism information under or in response to one or more conditions. For example, microorganisms in an immobilized sample may be tested for antimicrobial agent susceptibility by placing the microorganisms in an antimicrobial agent condition, such as by adding an antibiotic to a pre-immobilization or an immobilized microorganism sample. The system is capable of determining at least one of microorganism growth, antimicrobial agent susceptibility, and antimicrobial agent resistance. Identification and evaluation may comprise any of a single variable, single-factorial, multi-variable or multi-factorial analysis.

Immobilization and Tracking

Various aspects, methods, compositions and systems for immobilizing a microorganism as described herein facilitate tracking of the microorganism throughout a microorganism information acquisition process. In various embodiments, the restriction of microorganism movement permits repeated measurement of the microorganism and acquisition of microorganism information over time in a manner compatible with statistical confidence that microorganism information is obtained from the same microorganism at a second acquisition time as at a first acquisition time. Expressed differently, restriction of microorganism movement may facilitate tracking of an individual microorganism over a period of time. The relative degree of restricted movement required for tracking may be proportional to (directly or inversely) various factors, including the speed with which microorganism information is acquired, the sample complexity and/or microorganism density, the resolution of the detector, the rate of microorganism growth, the time period required for microorganism detection, and the like. For example, little to no microorganism immobilization would be required within a sample volume for systems in which the entire sample volume could be simultaneously assessed in real time and at high resolution. Conversely, for samples with high microorganism density, a relatively high degree of restriction of movement or confinement would be required to facilitate microorganism tracking.

Tracking of both a founder cell and daughter cell progeny in a sample during growth is provided for purposes such as assessing a clone's response to a stimulus. The analysis of individual clones as provided herein enables the construction of a population model and full characterization of the population at the level of each individual constituent. A population's response to stimulus is often non-Gaussian containing heterogeneity. In contrast, methods that measure and average the entire population's (all clones in bulk) response to stimulus (such as antibiotic agent exposure) cannot deduce the heterogeneity present in the population without inferring a population model. Thus, tracking of all individual cells, their progeny cells, and the corresponding clones is foundational to actual characterization of a population response to stimulus in a manner that can accurately account for a heterogeneous population.

Regardless of whether an initial capture step is performed independently of immobilization, immobilization serves a further benefit of facilitating analysis of biological samples with extremely low cell densities as well as analysis of biological samples with very high microorganism densities. In any clinical sample analysis, a high confidence conclusion can only be made based on observation of a sufficiently large sample size. In the context of microorganism identification, AST analysis, and MIC determination, the necessary sample size can be, for example, in the range of 10-50 microorganisms. For certain biological sample types with very low cell densities, a substantial portion of the sample volume may need to be analyzed to characterize a sufficiently large population of microorganisms to generate a meaningful analytical conclusion.

For optically-based detection methods and systems applied to large samples or samples with very low sample concentrations, rapid microorganism detection and growth determination requires microscopic analysis of many fields of view or optical slices of a sample volume. Microscopic observation of objects comprising a minor fraction of the total sample space is a challenge further complicated by object movement within the sample space. On the other hand, immobilization of the microorganisms in the sample space dramatically improves the ability of a system to scan a maximum volume of a sample space with movement of the detector relative to the sample, facilitating effective scanning while also providing the capacity to systematically return to a previously detected sample object repeatedly in the time frame of an assay and make a confident assessment that any change observed at the revisited location is an actual change in an attribute of the object.

Immobilization can provide similar benefits for very high-density samples, though those benefits may be realized in different aspects. In a high-density sample context, immobilization may, among various other benefits described herein, serve to prevent a proportion of microorganisms in a sample from physically interfering with each other in a period of time over which the microorganisms grow, thereby permitting a detection system to continue to be able to distinguish those microorganisms from one another and thereby track the microorganisms, similarly ascribing any change in a measured attributed at the location of the microorganism to growth of the microorganism. Explained differently, an immobilizing medium may prevent a first microorganism from coalescing with or becoming indistinguishable by the detection system from a second, adjacent microorganism that is not clonally derived from or progeny of the first microorganism. In various embodiments, immobilization can prevent more than about 50%, or more than about 60%, or more than about 70%, or more than about 80%, or more than about 90%, or more than about 95% of the CFUs in a sample from physically interfering with one another in the time frame following immobilization in which detection of growth is performed. Similarly, an immobilizing medium may prevent an attribute of a second microorganism from influencing a determination of growth of the microorganism by the detection system. Preventing an attribute of a microorganism from influencing a determination of growth can comprise creation of a discrete microenvironment that prevents a microorganism from influencing the growth of a second microorganism, or it can comprise reducing the interaction or influence such that a determination of growth can still be made (i.e., the first microorganism can still be distinguished and growth measured, even though the rate of growth might be influenced by a second microorganism). Expressed differently, an immobilizing medium may be suitable to prevent microorganism information for a first microorganism at a first location from influencing the detection of second microorganism information for a second microorganism at a second location.

Maintaining distinguishable microorganisms throughout an assay period by immobilization of a high density sample for even a small proportion of the sample microorganisms may permit a clinically meaningful conclusion to be drawn from the assay, assuming that the observed microorganisms comprise a representative subpopulation of the sample and that the growth patterns or responses of those microorganisms to a test condition under conditions of the assay (i.e., the sample microorganism density) can predict a clinically relevant conclusion (e.g., antibiotic susceptibility).

As described herein, the power of quantum microbiology (i.e., the ability to perform microbiological evaluations for individual microorganisms or clones as the fundamental unit for which microorganism data is obtained) allows individual clone growth dynamics—dynamics that would be completely masked in traditional macroscopic end-point assays or bulk culture assays—to be assessed in a variety of culture conditions that accommodate a wider range of sample types compared to traditional methods.

### EXAMPLES

### Example 1

### Same-Day Blood Culture with Digital Microscopy

### Background

Patients who acquire a bloodstream infection must begin adequate antibiotic therapy as quickly as possible. For critically ill patients, resistance can render initial therapy ineffective, delaying the start of effective antimicrobial therapy. The requirement for overnight culture creates an unacceptable delay. Delay also prolongs exposure to broad-spectrum empiric therapy, creating selective pressure favoring emergent resistance. Systems and methods in accordance with various embodiments of the present disclosure, such as automated digital microscopy detection systems, have the potential to reduce turnaround time by rapidly analyzing live bacteria extracted directly from a clinical specimen, eliminating the need for colony isolates. Microorganism immobilization can facilitate automated microorganism detection.

A study was performed to evaluate the ability of an automated system to perform same-day analysis of live organisms extracted directly from blood. The tests used two of the most common ICU pathogens, Staphylococcus aureus (SA) and Pseudomonas aeruginosa (PA).

### Methods

Simulated blood specimens were used. Simulated blood specimens were produced by spiking SA, PA, and non-target bacilli species isolates into 10 mL volumes of blood from two short-fill CPD blood bank bags. A total of 29 simulated blood samples were produced, each having a microorganism concentration of approximately 5 CFU/mL of bacterial target species, confirmed by quantitative culture. Spiked isolates used to produce the simulated blood samples included 14 Staphylococcus aureus (SA), 3 Pseudomonas aeruginosa (PA), and 12 non-target Gram-negative bacilli species. Twenty additional control samples were produced which contained no spikes. Each sample was diluted with 30 mL of modified tryptic soy broth medium to promote growth, followed by a 4-hour incubation at 35° C.

Following the 4-hour incubation period, samples were centrifuged briefly. The resulting microorganism cells pellets were resuspended in an electrokinetic concentration buffer to produce 1 mL samples for introduction into the detection system. Microorganism samples comprising 20 μL aliquots were pipetted into 14 microchannel flowcells. A 5-minute low-voltage electrokinetic capture was performed to concentrate the microorganisms on to a detection surface associated with each flowcell. The capture surface comprised a capture coating to immobilize the bacterial cells.

Liquid (40° C.) MHA, with and without antimicrobial agents, was then exchanged through each flowcell channel and immobilized, as described below. Separate channels received immobilizing medium containing antibiotics, which included the following antibiotics and concentrations: 32 μg/mL amikacin (AMK), 8 μg/mL imipenem (IPM), 6 μg/mL cefoxitin (FOX), or 0.5 μg/mL clindamycin (CLI) (all antibiotics were obtained from Sigma-Aldrich). A cooling step was then performed. This induced a phase change of the agar-immobilizing agent and immobilization of the captured microorganisms. Immobilized microorganism samples were incubated at 35° C. Sample imaging was then performed using an automated digital microscopy microorganism detection system.

The automated digital microscopy microorganism detection system acquired darkfield images every 10 minutes for three hours. The detection system applied identification algorithms to each individual immobilized cell that exhibited growth. Six flowcell channels provided data for ID algorithms to score individual organisms and their progeny clones. Identification algorithm variables included cell morphology, clone growth morphology, clone growth rate, and other growth-related factors. Controls included quantitative culturing, disk diffusion tests for isolate resistance phenotype, and 20 blood samples without spikes.

### Results

Culture confirmed that normal growth occurred in the simulated blood samples. Microorganism detection was performed for samples with greater than or equal to four growing clones. Recovery yielded SA GC counts that exceeded CFU as determined by culturing because of near-complete clump disruption in most samples. Counting combined results in multiple channels when appropriate. Identification was performed for samples with greater than or equal to 40 growing clones, and phenotype tests were performed for samples with greater than or equal to 40 growing clones. The detection system detected growth in 29/29 spiked samples and no growth in 20/20 non-spiked controls. Growth sufficient for identification occurred in 23/29 samples in the fixed 4-hour growth period. Four SA samples clumped excessively, precluding identification scoring. Two PA samples grew too slowly (here, not greater than 1.1 div/hr) to achieve 40 growing clones in a desired growth period. SA growth rates were greater than or equal to 1.5 div/hr. The detection system identified 1/1 PA and 10/10 SA. One false PA identification occurred out of 22 non-target samples to yield 100% sensitivity and 97% specificity. The false ID was attributable to a known imaging aberration, later corrected.

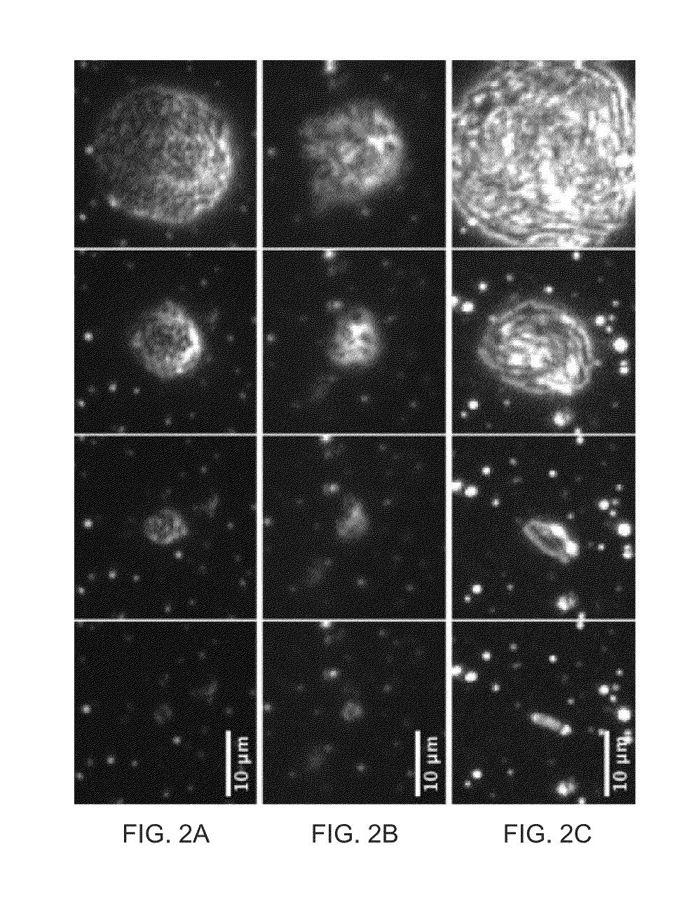

FIGS. 2A-2C illustrate examples of darkfield images of immobilized microorganism samples acquired using a microorganism detection system over a period of 3 hours (time points at 0, 60, 120 and 180 minutes) of clone growth in immobilizing medium for SA without drug (FIG. 2A, no antibiotic), SA in 6 μg/mL FOX (FIG. 2B, cefoxitin) growth indicating a methicillin-resistant phenotype, and for a Gram-negative rod (E. coli) without drug (FIG. 2C) for morphology comparison. Non-growing particles are assumed to be debris.

The immobilization and automated digital microscopy detection system identified drug resistance in 19/20 adequate samples with one false methicillin-susceptible SA determination. Thus, drug resistance phenotyping was performed with 89% sensitivity and 100% specificity. Table 1 summarizes SA data for overall concordance with comparator results.

TABLE 1
Identification of S. aureus drug resistance phenotypes using automated
digital microscopy detection system analysis.

| | | | |
|---|---|---|---|
| S. aureus | True Neg | True Pos | Accuracy |

IDENTIFICATION (Adequate Growth N = 23)

| | | | |
|---|---|---|---|
| Positive | 0 | 10 | Sens 100% (CI 66-100%) |
| Negative | 23 | 0 | Spec 100% (CI 72-100%) |

PHENOTYPE: MRSA (Adequate Growth N = 10)

| | | | |
|---|---|---|---|
| Positive | 0 | 4 | Sens 80% (CI 30-100%) |
| Negative | 5 | 1 | Spec 100% (CI 46-100%) |

PHENOTYPE: CLI-R (Adequate Growth N = 10)

| | | | |
|---|---|---|---|
| Positive | 0 | 4 | Sens 100% (CI 40-100%) |
| Negative | 6 | 0 | Spec 100% (CI 52-100%) |

Starting with simulated blood samples, microorganism capture and immobilization facilitated microorganism detection using automated microscopy. Target pathogens were successfully identified and drug resistance phenotypes detected for a major species of live bacterial cells extracted directly from a small volume of simulated bacteremic blood, all within 8 hours. This diagnostic analysis using individual immobilized microorganism cells enables rapid turnaround without first requiring colony isolates.

Thus, application of systems and methods in accordance with various embodiments of the present disclosure enables immobilization of live microorganisms for successful detection using an automated digital microscopy detection system.

### Example 2

### Comparison of Plating and Biosensor Immobilization Methods of Cell Enumeration

### Methods

Dilution series of bacteria were plated using various quantitative procedures, including pour plates, streak plates, and a quantitative liquid plating method, to determine the accuracy and dynamic range of viable microorganism enumeration. Growth detection was performed the same samples immobilized in a three-dimensional space, coupled to a microfluidic detection device with microscopy and automated image analysis. The accuracy and dynamic range of quantitation of the former compared to the latter was assessed.

Bacterial strains of Escherichia coli (ATCC-25922; American Type Culture Collection (ATCC), Manassas, Va.) (Ecol) and Acinetobacter baumannii (ATCC-19606; ATCC) (Abau) were grown to obtain colonies on solid media plates (TSA II Blood Agar, Becton-Dickinson (BD), Franklin Lakes, N.J.). Colonies of bacteria were taken from these plates and suspended in liquid culture medium (Cation-Adjusted Mueller-Hinton Broth, CA-MHB, BD) to a predetermined density as measured by a bacteriological nephelometer (Densi-Check, BioMerieux Inc., Durham, N.C.). The initial suspension was diluted in CA-MHB in 10-fold dilution series to obtain suspensions ranging from 10 to 1 billion (1×109) colony forming units per mL (CFU/mL).

Pour plates were prepared by diluting 1 mL of each of the 1×101, 1×102, 1×103, 1×104, and 1×105 cell concentrations into 9 mL of liquid (molten) Cation-Adjusted Mueller-Hinton agar (CA-MHA) containing 0.944% Noble agar (Sigma Aldrich). The entire volume of each was poured into an empty 100 mm Petri dish. The plates were cooled to allow the agar to harden (0.850% final agar concentration), and incubated overnight at 35° C. Streak plates were prepared by spreading 50 μL each of 1×102, 1×103, 1×104, 1×105, and 1×106 cell concentrations onto a blood agar plate using a 10 μL loop. The plates were allowed to dry at ambient temperature, and then incubated overnight at 35° C. Quantitative liquid plating was performed by adding 50 μL each of 1×102, 1×103, 1×104, 1×105, and 1×106 cell concentrations to blood agar plates in several dispersed drops and spreading the liquid across the top surface of the agar using gravity. The plates were allowed to dry at ambient temperature, and then incubated overnight at 35° C. The number of colonies was counted for each plate following overnight incubation.

For biosensor immobilization, 180 μL aliquots of liquid CA-MHA containing 0.944% agar as an immobilizing agent were placed in wells of a round bottom multi-well plate maintained at 45° C. Bacteria from each suspension were diluted with 9 parts of the CA-MHA by mixing a 20 μL aliquot of each cells stock produce pre-immobilization samples with 1×104 to 1×107 CFU/mL cell densities in CA-MHA with 0.850% agar.

These pre-immobilization samples were added to flowcells in a microfluidic detection device for imaging. The microfluidic detection device consisted of a series of flowcell channels having unique pipet-interface entry ports and waste (exit) ports for each channel. The biosensor device was maintained at 40° C., and 100 uL of pre-immobilization sample was pipetted into each flowcell channel immediately after mixing, with sufficient excess to fill the channel and plug the ports. The pre-immobilization samples were cooled below the agar gelling point in order to induce a phase change of the agar-immobilizing agent and immobilize the suspended bacteria. Ports were covered with mineral oil to prevent evaporation of the solidified medium during the growth period.

The biosensor device was then imaged using darkfield illumination in an automated imaging system that controlled illumination, stage motion, and focus. Digital images were taken at each site, focal plane, and time in 12-bit grayscale and stored to a computer hard drive. The imaging system was programmed to scan a single site of each flowcell (i.e., a single field of view) with eight different focal positions covering the entire z-axis span of the flowcell. Each field of view covered approximately 592×444 microns, with a column height of 300 microns on the z-axis, resulting in a total imaged volume of 0.0789 μL (a FOV column volume). Within this format, each bacterium observed in a field of view represented ˜13,000 (1.3×104) CFU/mL. The bacteria were imaged at T=0 and every 10 minutes after that for a total of 4 hours (T=240).

For analysis, images from each site were stacked to create a time-lapse series that allowed determination of bacterial growth over time. Growth was observed to start from a single colony forming unity (CFU) with size and brightness increasing over time. Clones growing above or below the focal plane could be observed in multiple stacks at lower intensity, allowing discrimination of each immobilized clone. Clones could be discriminated sufficiently to allow enumeration given sufficient distance in x-, y-, or z-axis dimensions, typically approximately 1 cell diameter. The total number of growing bacteria in the site was multiplied by 1.3×104 to calculate the concentration in the sample.

### Results

Results are shown in Tables 2 and 3. Using pour plate, spread plate, and liquid plate methods, relatively low concentrations were quantifiable while higher concentrations required sample dilution to achieve accuracy. Typically, colonies ranging up to 200 could be discriminated on a 100 mm petri dish. Below counts of 20, the quantitation was less accurate, producing an accurate dynamic range of about 10-fold (20 to 200 colonies). This small dynamic range required significant dilution of many samples and required that several dilutions be plated to enumerate bacteria from samples of unknown concentration. Quantities for each plating method were within ½-log of each other in most cases. Obligate aerobic bacteria were undercounted using the pour plate method.

The microscopic growth method showed a countable range of 2.6×105 to 2.6×107 CFU/mL (20 to 2000 growing clones per field of view), using a single field of view column (multiple focal planes). Thus, microscopic imaging provides an ability to enumerate much higher cell densities then plating methods relying on macroscopic colony evaluation. The wider dynamic range can accommodate a greater input concentration variation without requiring extra handling. Furthermore, by imaging additional fields of view (up to 20 per flowcell) the lower end of the dynamic range could be extended by more than 10-fold (1.3×103 CFU/mL with 20 clones in 20 fields of view).

The 1×108 inoculum concentration produced many unresolvable clones that interfered with accurate enumeration. The estimated upper limit of clones in a FOV column for enumeration purposes is around 2000. However, a large number of clones could be distinguished over the course of the four hour growth period, and even for samples with microorganism densities similar to this inoculum concentration, analysis of microorganism viability and susceptibility could be performed based on the number of resolvable microorganisms.

In summary, the ability to analyze growth using microscopy and computer image analysis software allowed enumeration in a much shorter time period (approximately 4 hours) than is required for any of the traditional plating methods, which require overnight or longer incubation periods to produce countable macroscopic colonies.

TABLE 2
Comparison of Microscopic Growth and Various Plating Methods for
Enumeration of Escherichia coli (ATCC-25922).

| | Pour | | Spread | Spread | Liquid | Liquid | Microscopic | |
|---|---|---|---|---|---|---|---|---|
| Inoculum | Plate | Pour Plate | Plate | Plate | Plate | Plate | Count per | Microscopic |
| Conc. | Count | Conc. | Count | Conc. | Count | Conc. | FOV | Concentration |
| 1.0 × 10¹ | N/A | N/A | N/A | N/A | N/A | N/A | N/A | N/A |
| 1.0 × 10² | 98 | 9.80 × 10¹ | 6 | 1.20 × 10² | 6 | 1.20 × 10² | N/A | N/A |
| 1.0 × 10³ | TMTC | N/A | 61 | 1.22 × 10³ | 75 | 1.50 × 10³ | N/A | N/A |
| 1.0 × 10⁴ | TMTC | N/A | TMTC | N/A | TMTC | N/A | 0 | N/A |
| 1.0 × 10⁵ | TMTC | N/A | TMTC | N/A | TMTC | N/A | 13 | 1.69 × 10⁵ |
| 1.0 × 10⁶ | TMTC | N/A | TMTC | N/A | TMTC | N/A | 101 | 1.31 × 10⁶ |
| 1.0 × 10⁷ | N/A | N/A | N/A | N/A | N/A | N/A | 880 | 1.14 × 10⁷ |
| 1.0 × 10⁸ | N/A | N/A | N/A | N/A | N/A | N/A | TMTC | N/A |

TABLE 3
Comparison of Microscopic Growth and Various Plating Methods for
Enumeration of Acinetobacter baumannii (ATCC-19606).

| | Pour* | | Spread | Spread | Liquid | Liquid | Microscopic | |
|---|---|---|---|---|---|---|---|---|
| Inoculum | Plate | Pour Plate | Plate | Plate | Plate | Plate | Count per | Microscopic |
| Conc. | Count | Conc. | Count | Conc. | Count | Conc. | FOV | Concentration |
| 1.0 × 10¹ | 9 | 9.00 × 10⁰ | N/A | N/A | N/A | N/A | N/A | N/A |
| 1.0 × 10² | 28 | 2.80 × 10¹ | 6 | 1.20 × 10² | 10 | 2.00 × 10² | N/A | N/A |
| 1.0 × 10³ | TMTC | N/A | 68 | 1.36 × 10³ | 51 | 1.02 × 10³ | N/A | N/A |
| 1.0 × 10⁴ | TMTC | N/A | TMTC | N/A | TMTC | N/A | 0 | N/A |
| 1.0 × 10⁵ | TMTC | N/A | TMTC | N/A | TMTC | N/A | 1 | 1.30 × 10⁴ |
| 1.0 × 10⁶ | TMTC | N/A | TMTC | N/A | TMTC | N/A | 30 | 3.90 × 10⁵ |
| 1.0 × 10⁷ | N/A | N/A | N/A | N/A | N/A | N/A | 305 | 3.97 × 10⁶ |
| 1.0 × 10⁸ | N/A | N/A | N/A | N/A | N/A | N/A | N/A | N/A |

*High count values for pour plates likely reflects artifact due to counting of bubbles produced by pour plate technique.

Certain plating strategies are not compatible with all bacteria types, particularly if the bacteria require oxygen or if they were prone to spreading significantly on the agar plate surface. For this reason, a particular type of bacteria may require a particular quantitation medium and plating strategy to obtain accurate results. Samples of unknown composition may require multiple rounds of optimization to accurately assess the quantity of bacteria. The microscopic method may also be incompatible with certain types of bacteria since the medium must support growth of the organism, allow the bacteria to be immobilized, and provide sufficient optical clarity to allow high resolution imaging. However, for most human pathogenic bacteria, the common growth medium described above (CA-MHB with 0.85% agar) allows visualization of growth. This method has the advantage of being able to enumerate bacteria that would not grow to produce visible colonies on a plate due to environmental restrictions and the method allows enumeration of bacteria that swim or swarm on surfaces over time.

### Example 3

### Comparison of Immobilizing Agent Concentrations on Immobilization and Microscopic Biosensor Enumeration

### Methods

Heavy suspensions of Ecol 25922 and Paer 27853 were prepared in CA-MHB from fresh overnight blood agar plates. Heavy suspensions were diluted in normal saline to produce 0.5 McFarland suspensions, noting the volumes of each suspension required to produce 1×108 CFU/mL suspensions. For each strain, a 1×109 cfu/mL starting suspension in CA-MHB was then made. These starting suspensions were diluted in series to create 1×108 to 1×105 cfu/mL suspensions as 10× microorganism suspension stocks.

Three different immobilizing agent concentrations were tested. Liquid (molten) CA-MHA stock solutions were prepared at with the 1.44%, 0.944%, and 0.470% agar concentrations. A round bottom 96-well plate was placed in a plate heater set to 47° C., and 180 μL volumes of CA-MHA stock solutions were aliquoted into 5 wells for each stock. 20 μL of each of the microorganism suspension stocks were diluted into the CA-MHA aliquots and mixed to produce pre-immobilization samples with microorganism concentrations of 1×108 to 1×104 cfu/mL and agar concentrations of 1.30%, 0.850%, and 0.423%. Immediately after mixing, 100 μL of each pre-immobilization sample was injected into a microchannel flowcell of a biosensor device maintained at 40° C. After all pre-immobilization samples were introduced the biosensor was cooled to induce a phase change of the agar and immobilization of the sample microorganisms. The flowcell ports were overlaid with mineral oil to seal them, and the biosensor placed in a detection system for microorganism detection and growth analysis.

The biosensor device was then imaged using darkfield illumination in an automated imaging system that controlled illumination, stage motion, and focus. Digital images were taken at each site, focal plane, and time in 12-bit grayscale and stored to a computer hard drive. The imaging system was programmed to scan a single site of each flowcell (i.e., a single field of view) with eight different focal positions covering the entire z-axis span of the flowcell. Each field of view covered approximately 592×444 microns, with a column height of 300 microns on the z-axis, resulting in a total imaged volume of 0.0789 μL (a FOV column volume). Within this format, each bacterium observed in a field of view represented 13,000 (1.3×104) CFU/mL. The bacteria were imaged at T=0 and every 10 minutes after that for a total of 4 hours (T=240).

### Results

As shown in Tables 4 and 5, at 0.423% for both Ecol and Paer, a small number of cells could be visualized at various time points in the experiments. However, the clones were not sufficiently immobilized for accurate resolution and enumeration. Likewise, clones could not be tracked, nor could a determination of growth be made at this immobilizing agent concentration. At the 1.30% agar immobilizing agent concentration, clones could be distinguished and counted at 1×107, 1×106, and 1×105 (Paer) cfu/mL concentrations. Similar results were obtained for Ecol using a 0.850% agar immobilizing agent concentration. Numerous discrete clones could be resolved at the 1×108 cfu/mL concentrations for both strains with the 1.30% agar medium and for Ecol with the 0.850% agar medium, but enumeration could not be performed due to physical interference of a significant proportion of the clones in the FOV column. Nonetheless, many clones could be distinguished throughout the four-hour growth period with detectable growth, and antimicrobial agent susceptibility testing with complex samples may be feasible at very high cell densities.

TABLE 4
Comparison of Microscopic Enumeration of Escherichia coli
(ATCC-25922) in MHA at Three Immobilizing Agent Concentrations.

| | Agar | | |
|---|---|---|---|
| Inoculum Conc | concentration | Clone Count | GC/mL (count × |
| (cfu/mL) | (% w/v) | per FOV | 1.3 × 10⁴ |
| 1.00 × 10⁸ | 1.30 | >2000 | >2.6 × 10⁷ |
| 1.00 × 10⁷ | 1.30 | 496 | 6.45E+06 |
| 1.00 × 10⁶ | 1.30 | 111 | 1.44E+06 |
| 1.00 × 10⁵ | 1.30 | 0 | <1 × 10⁴ |
| 1.00 × 10⁴ | 1.30 | 0 | <1 × 10⁴ |
| 1.00 × 10⁸ | 0.850 | >2000 | >2.6 × 10⁷ |
| 1.00 × 10⁷ | 0.850 | 587 | 7.63E+06 |
| 1.00 × 10⁶ | 0.850 | 20 | 3.25E+05 |
| 1.00 × 10⁵ | 0.850 | 12 | 1.56E+05 |
| 1.00 × 10⁴ | 0.850 | 6 | 7.80E+04 |
| 1.00 × 10⁸ | 0.423 | N/A | N/A |
| 1.00 × 10⁷ | 0.423 | N/A | N/A |
| 1.00 × 10⁶ | 0.423 | N/A | N/A |
| 1.00 × 10⁵ | 0.423 | N/A | N/A |
| 1.00 × 10⁴ | 0.423 | N/A | N/A |

TABLE 5
Comparison of Microscopic Enumeration of P. aeruginosa (ATCC-27853)
in MHA at Two Immobilizing Agent Concentrations.

| | Agar | | |
|---|---|---|---|
| Inoculum Conc | concentration | Clone Count | GC/mL (count × |
| (cfu/mL) | (% w/v) | per FOV | 1.3 × 10⁴ |
| 1.00 × 10⁸ | 1.30 | >2000 | >2.6 × 10⁷ |
| 1.00 × 10⁷ | 1.30 | 368 | 4.78E+06 |
| 1.00 × 10⁶ | 1.30 | 162 | 2.11E+06 |
| 1.00 × 10⁵ | 1.30 | 2 | 2.60E+04 |
| 1.00 × 10⁴ | 1.30 | 0 | <1 × 10⁴ |
| 1.00 × 10⁸ | 0.423 | N/A | N/A |
| 1.00 × 10⁷ | 0.423 | N/A | N/A |
| 1.00 × 10⁶ | 0.423 | N/A | N/A |
| 1.00 × 10⁵ | 0.423 | N/A | N/A |
| 1.00 × 10⁴ | 0.423 | N/A | N/A |

### Example 4

### Antibiotic Susceptibility Interference Testing

### Background

Certain antibiotic resistance mechanisms can be mediated by enzymes released from the outer surface of bacteria, which then act on antibiotic molecules in the surrounding medium. It is known that this mechanism can result in an apparent increased resistance to antibiotics for certain bacteria types when they are grown in a mixed culture with a resistant organism. In this context the actual antibiotic concentration is reduced by the enzyme rather than resistance being acquired by the sensitive strain.

Several nutrient agar plating methods employing antibiotics, other chemicals, and one or more species of bacteria have been devised to study these mechanisms. The most basic of these tests, the disk-diffusion method, employs a single test strain of bacteria which is used to create a uniform layer or lawn when grown. A disk-containing antibiotic is placed on the inoculated plate and the plate is then incubated to grow the lawn. In cases where the strain is sensitive to the antibiotic, a zone of no growth is observed around the disk. The distance from the disk to the edge of this zone is an indicator of the level of resistance to the antibiotic present in the disk, based on how far the antibiotic can diffuse into the nutrient medium. A variation on this test is to employ a second disk or drop of fluid near the antibiotic disk that contains a chemical that can affect the antibiotic response. Differences between the zone in areas near the chemical and away from the chemical can indicate the type of resistance mechanism at work in the test. In this case, the chelating agent ethylenediamine tetraacetic acid (EDTA) (Sigma-Aldrich) can be used to test for the presence of a class of antibiotic resistance enzyme called metallo-beta-lactamase, which is inhibited by EDTA. The positive result for this test is a larger zone of inhibition near the EDTA indicating reduced effectiveness of the enzyme. A more complex test called the Hodge test can be used to indicate antibiotic resistance conferred to an antibiotic-sensitive sentinel strain in the presence of an antibiotic-resistant test strain. The sentinel strain is grown as described above for the disk diffusion method and the test strain is streaked from the disk to the outer edge of the plate. A positive result in this test is indicated by the sentinel strain growing closer to the disk in the presence of the test strain than in other areas. This mechanism for this response is the release of soluble enzyme, which can diffuse away from the test strain line and effectively reduce the antibiotic concentration in those areas, allowing the sentinel strain to grow. A variation on this method is to perform the disk diffusion test on a mixed lawn and look for the presence of the sentinel strain in the zone of inhibition after growth using an indicator method.

### Method

Mixed species disk diffusion tests were performed to evaluate post-growth presence/absence of E. coli 25922 (Ecol) in an inhibition zone due to the presence of P. aeruginosa 519749 (Paer). 0.5 McFarland suspensions of Ecol and Paer were prepared from fresh overnight blood agar plates. Lawn plates of each single organism were prepared by spreading each suspension on two blood agar plates per isolate. A 1:1 mix of Ecol and Paer was prepared and used create two lawn plates of the mixed isolates. An IMP 10 disk was placed on each isolate lawn plate and the mixed isolate plate, and the plates were incubated overnight at 35° C. Each single isolate plate was observed for appropriate sensitivity and resistance to each antibiotic, and the mixed isolate plate was observed for evidence of Ecol growing in the inhibition zone using oxidase (Ecol negative, Paer positive) and indole tests (Ecol positive, Paer negative).

An EDTA-IPM disk diffusion test was performed to assess post-growth presence/absence for increased inhibition of metallo-beta-lactamase by EDTA. A 0.5 McFarland suspension of Paer was prepared from a fresh overnight blood agar plate and used to prepare a lawn plate on blood agar medium. A 5 μL drop of 5 mM EDTA was placed on the plate just away from the center and the location of the drop marked. An IPM 10 disk was placed near the location of the EDTA drop, and the plates were incubated overnight at 35° C. Plates were observed for increased inhibition near the EDTA drop indicating metallo-beta-lactamase, which is inhibited by EDTA.

A mixed species diffusion variation of Hodge test using a biosensor with immobilized cells and microscopic detection was performed using the same bacterial strains. Heavy suspensions of Ecol and Paer were prepared in L-histidine buffer from fresh overnight blood agar plates. Heavy suspensions were diluted in normal saline to produce 0.5 McFarland suspensions, noting the volumes of each suspension required and using the same volumes to produce 108 cfu/mL suspensions of each strain in L-histidine. These suspensions were further diluted to create 2×106 and 2×105 cfu/mL suspensions for heavy and moderate density working samples. The working samples were then diluted 1:1 in 2 mM L-DOPA for EKC.

The strains were next introduced to a biosensor for EKC, immobilization, and detection. Paer suspensions were loaded first, placing 5 uL in the exit port side (to maintain the resistant Paer isolate in one half of each channel) of each biosensor flowcell channel to cover one half of the flowcell. Six flowcells were loaded with each concentration. EKC was performed for 5 minutes at 1.5V, and then all flowcells were washed with two aliquots of 160 μL 1 mM L-DOPA (from the entry port). Ecol suspensions were next loaded, introducing 20 μL of cells suspension from the entry port to cover the full flowcell. Six flowcells were loaded with each concentration. EKC was performed for 5 minutes at 1.5V, and all flowcells washed with 160 μL of 1/10 MHB. The manner in which the cells were introduced and captures created a site comprising Ecol cells that were distal from any Paer cells (i.e., on the entry port side of the flowcell), as well as Ecol cells that were proximal to the Paer cells (i.e., on the exit port side of the flowcell).

Antibiotic media were prepared for immobilization and inhibition testing as follows. A 10×CAZ stock (160 μg/mL) and a 10×MEM stock (4 μg/mL) was prepared in MHB. An immobilization medium source plate was prepared in the detection system and maintained at 45° C. The source plate wells were loaded with 20 μL of the 10× stocks and MHB without antibiotic for growth controls. The sample cassette was placed in the detection system and maintained at 40° C. A 180 uL volume of 0.944% MHA was added to each well of the source plate and mixed, and 100 uL of pre-immobilization immobilizing medium (with an agar concentration of 0.850%) was withdrawn and introduced to a flowcell in accordance with the experimental design. The loaded biosensor was allowed to equilibrate for 5 minutes, and was then removed and cooled for 5 minutes to induce the agar immobilizing agent to change to solid phase. The flowcell ports were then sealed, and growth, imaging, and analysis were performed, with examination of each flowcell for growth of Paer, examination of the distal portion of each flowcell for growth of Ecol in the absence of Paer (distal sensitivity), and examination of the proximal portion of each flowcell for growth of Ecol in the presence of Paer (proximal resistance), indicative of a positive mixed species resistance due to diffusion of metallo-beta-lactamase.

### Results

In order to test for enzyme-mediated cross-species resistance effects, a method for was devised to test a sensitive strain, Escherichia coli ATCC 25922 (Ecol), in the presence of a resistant strain, Pseudomonas aeruginosa IHMA 519749 (Paer) in the immobilized format. The method involved first immobilizing the resistant strain on the surface of a portion of each test flowcell of the cassette (approximately half) and then immobilizing the sensitive (sentinel) strain on the surface of the full flowcell including the area containing the resistant strain. This strategy provides an internal control for treatment effects on the sentinel strain in an area far from the resistant strain along with an area where close-field effects can be observed in the same flowcell. Provided that the growth and morphology characteristics for the two strains are significantly different in growth control and antibiotic treated conditions, any effects of the resistant strain on the sentinel can be observed. The method also allows testing of different inoculum concentrations and ratios of the sentinel and resistant strain, which can provide information regarding how far enzymes diffuse during the assay in a similar way that the Hodge test provides this in plating assays.

The action of resistance to two different antibiotics, imipenem (IPM) and ceftazidime (CAZ), was tested for both strains using traditional plate-based methods of disk diffusion, EDTA-imipenem, and mixed species disk-diffusion. The Ecol strain was sensitive to both antibiotics in all plating-based tests demonstrating its utility as a sentinel. The Paer isolate showed high resistance to both antibiotics in all plating-based tests with no zone of inhibition observed. The EDTA-imipenem method indicated that the Paer expressed a metallo-beta-lactamase enzyme. The mixed disk-diffusion variation of the Hodge test indicated that Ecol could grow in the zone of inhibition in the presence of the Paer strain.

Test conditions were created in the biosensor format with immobilized cells covering the range of 10:1, 1:1, and 1:10 ratios of each strain in combined concentrations ranging from around 20 to 200 cells per field of view, allowing conditions with close and more distant ranges of clone growth. Each ratio and concentration was tested for untreated (i.e., no antibiotic), CAZ at 16 μg/mL and meropenem (MEM; Sigma-Aldrich) at 4 μg/mL. MEM is similar to IPM and was used since a liquid antibiotic stock of IPM was not available. Both antibiotics were used at concentrations in the range where Paer should grow normally (½ of the minimum inhibitory concentration) but Ecol should not grow.

A combination of visual and computer-assisted image analysis was used to compare time-lapse images that were taken every 10 minutes during 4 hours of growth in each of the conditions. The growth of both strains was apparent in the growth control conditions with the Paer starting as a dim rod growing approximately 20 to 30-fold over 4 hours and the Ecol starting as a brighter rod growing approximately 200 to 300-fold over 4 hours. Ratios of 1:1 for each strain at the 20 and 200 cells per field of view provided spacing as predicted such that clones in the higher concentration for Ecol typically were within a colony diameter of at least 1 Paer colony. With the ratio of 1 Ecol to 10 Paer, several Paer colonies were within 1 colony diameter of each Ecol colony. Cells were typically monodispersed and evenly spaced at the beginning of the run.

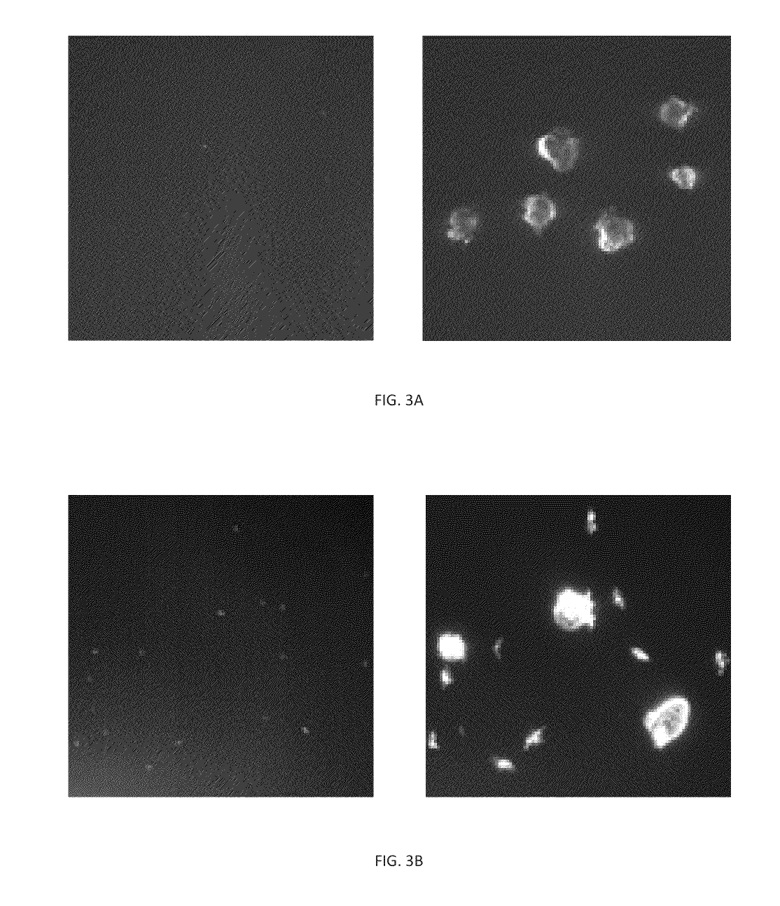

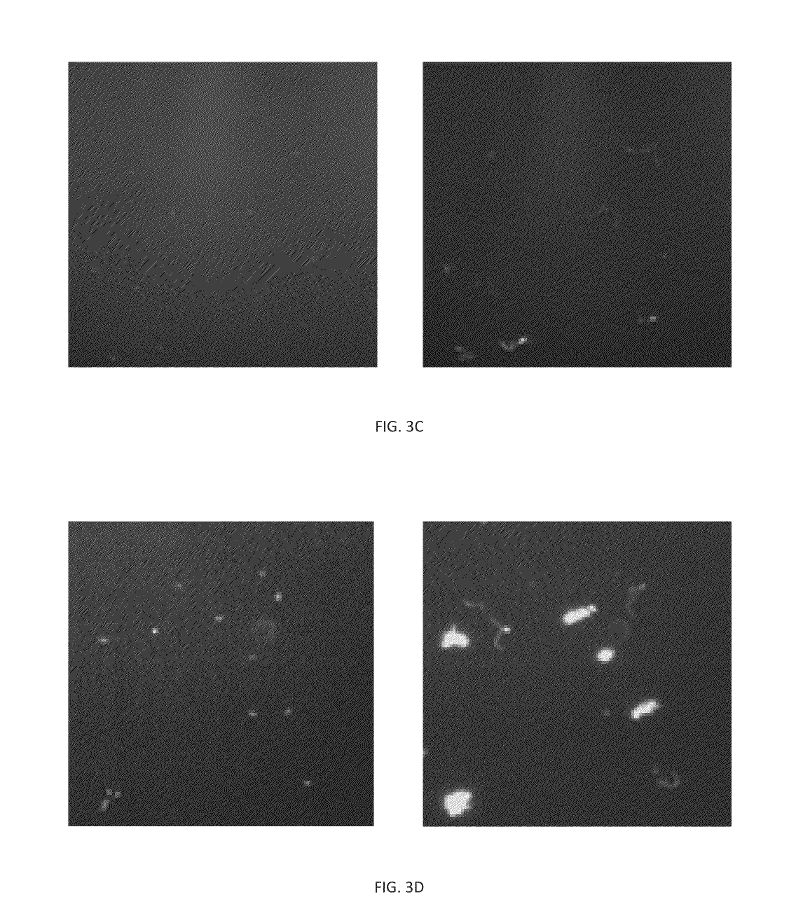

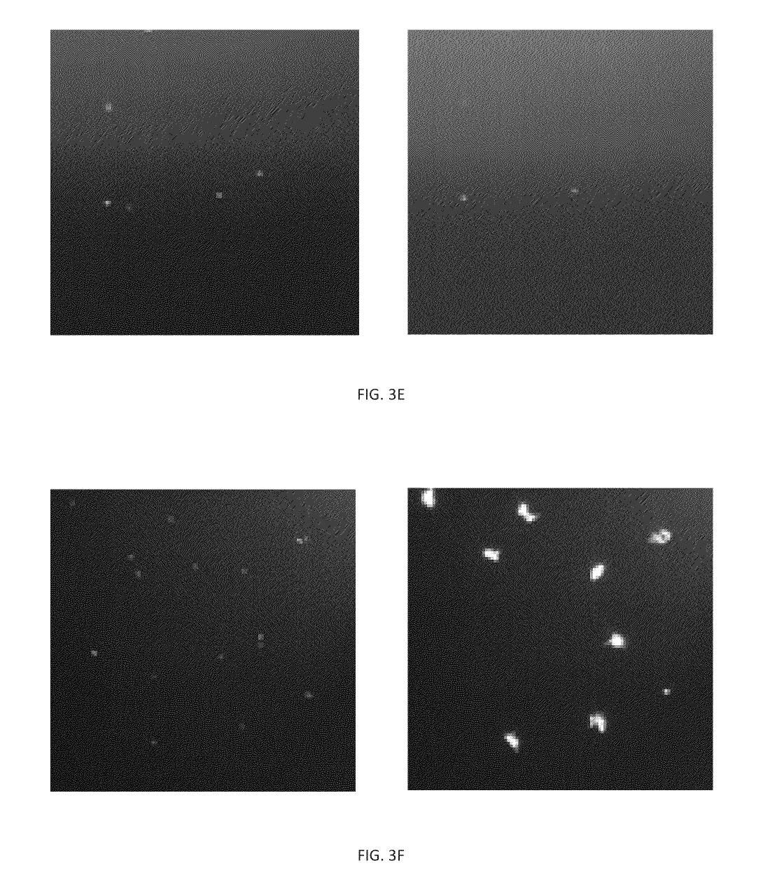

Results of the biosensor mixed species diffusion assay are shown in Table 6 and FIGS. 3A-3F. For each of FIGS. 3A-3F, the left hand panel shows an image acquired at time 0, and the right hand panel shows an image acquired after 260 minutes. FIG. 3A illustrates growth of Ecol cells in the distal site of a control flowcell (Flowcell #3). FIG. 3B illustrates growth of both Ecol and Paer in the proximal site of the same control flowcell. FIGS. 3C and 3E illustrate inhibition of Ecol in the distal site of a CAZ-treated flowcell (Flowcell #7) and the distal site of a MEM-treated flowcell (Flowcell #11), respectively. In the CAZ and MEM treated conditions, Ecol showed slight growth or elongation of the single cell with later fading and in many colonies complete lysis resulting in no detectable clone after 4 hours. In contrast and as illustrated in FIGS. 3D and 3F showing the proximal sites of the same CAZ- and MEM-treated flowcells (Flowcells #7 and #11, respectively), Paer appeared to grow with the same morphology and growth rate as the growth control condition in both antibiotics. In conditions where a single Ecol cell was surrounded by several Paer colonies, no difference in growth morphology or rate were observed, indicating that the Paer resistance did not affect the sensitivity of Ecol even at very close distances in the immobilized sample format, in contrast to the results obtained using a traditional plate-based mixed species assay in which a false positive resistance result was obtained due to growth of Ecol in the zone of inhibition.

TABLE 6
Results of biosensor mixed species diffusion assay.

| | | | Ecol | Ecol | Mixed species |
|---|---|---|---|---|---|
| | | | growth | growth in | resistance |
| Flowcell | | Paer | in distal | proximal | factor |
| # | Condition | growth? | site? | site? | diffusion? |
| 1 | Ecol 10⁶/ | Yes | Yes | Yes | Not |
| | Paer 10⁶ | | | | applicable |
| | Control | | | | |
| 2 | Ecol 10⁶/ | Yes | Yes | Yes | Not |
| | Paer 10⁵ | | | | applicable |
| | Control | | | | |
| 3 | Ecol 10⁵/ | Yes | Yes | Yes | Not |
| | Paer 10⁶ | | | | applicable |
| | Control | | | | |
| 4 | Ecol 10⁵/ | Yes | Yes | Yes | Not |
| | Paer 10⁵ | | | | applicable |
| | Control | | | | |
| 5 | Ecol 10⁶/ | Yes | No | No | No |
| | Paer 10⁶ | | | | |
| | CAZ 16 | | | | |
| 6 | Ecol 10⁶/ | Yes | No | No | No |
| | Paer 10⁵ | | | | |
| | CAZ 16 | | | | |
| 7 | Ecol 10⁵/ | Yes | No | No | No |
| | Paer 10⁶ | | | | |
| | CAZ 16 | | | | |
| 8 | Ecol 10⁵/ | Yes | No | No | No |
| | Paer 10⁵ | | | | |
| | CAZ 16 | | | | |
| 9 | Ecol 10⁶/ | Yes | No | No | No |
| | Paer 10⁶ | | | | |
| | MEM 4 | | | | |
| 10 | Ecol 10⁶/ | Yes | No | No | No |
| | Paer 10⁵ | | | | |
| | MEM 4 | | | | |
| 11 | Ecol 10⁵/ | Yes | No | No | No |
| | Paer 10⁶ | | | | |
| | MEM 4 | | | | |
| 12 | Ecol 10⁵/ | Yes | No | No | No |
| | Paer 10⁵ | | | | |
| | MEM 4 | | | | |

### Example 5

### Detection of Cross-Species Interference in Immobilized Format—Nutrient Exhaustion Methods

Different cell stock concentrations of E. coli ATCC 25922 between approximately 4×103 to 8×105 cells/mL were generated in EKC buffer to produce immobilized samples with microorganism concentrations ranging from 1 to 200 cells per field of view. Cells were immobilized by EKC and grown in MHA without antibiotics as described in Example 4. Analysis of growing clones and mean growth rates were performed as described in Example 1.

### Results

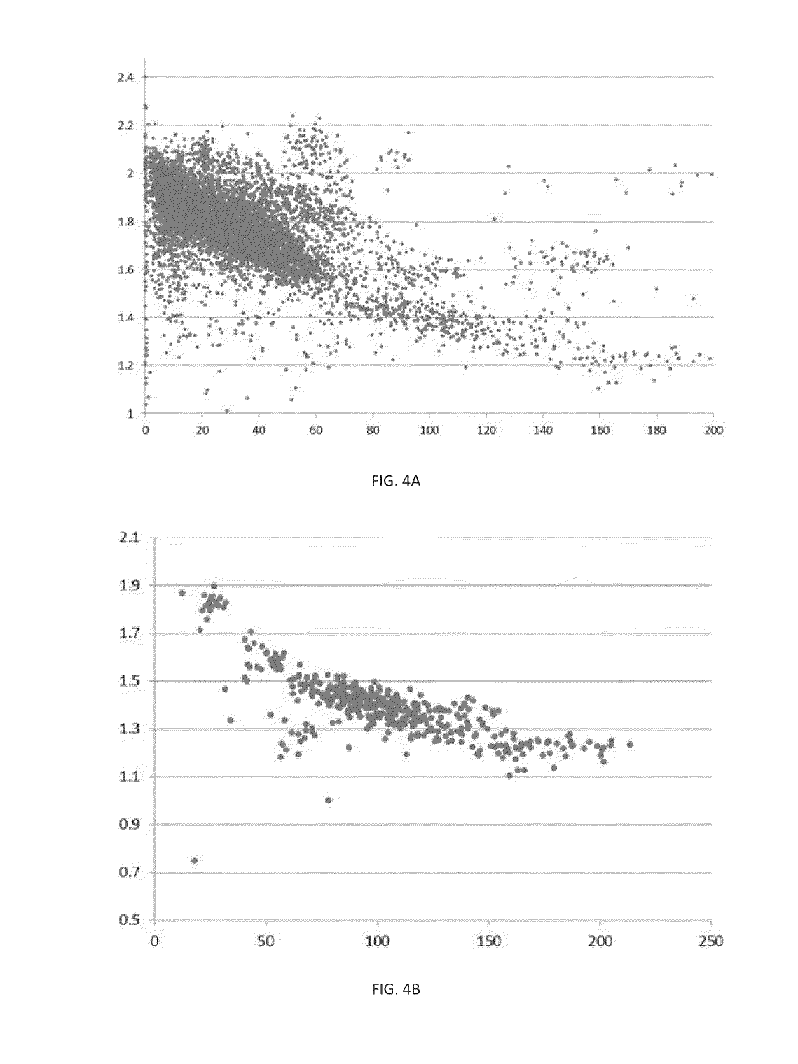

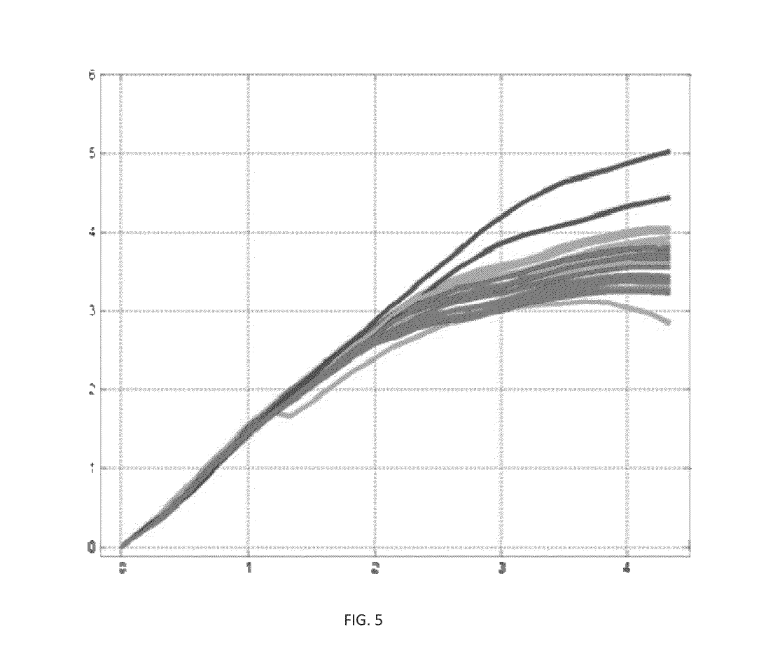

Growth data for individual clones of E. coli ATCC 25922 grown in immobilizing medium at various cell densities was collected from numerous experiments. Cell division rates were plotted against clone density. As illustrated in FIGS. 4A and 4B, clone density produced a decrease in the cell division rate, suggesting that increasing cell densities in the immobilizing medium may result in nutrient exhaustion or other competitive effects. Similarly, as illustrated in FIG. 5, plotting growth rate against time and categorizing the plotted rate by clone density (<50 clones/FOV, 50-100 clones/FOV, or >50 clones/FOV) showed a pattern of growth rates for individual clones slowing more dramatically and at earlier time points in the assays for the higher cell densities. Clones generally demonstrated consistent growth rates for the first three hours of a four hour growth period for samples with a density of <50 clones/FOV, while growth rates began to slow at around 2.5 hours for sample with 50-100 clones/FOV, and growth rates began to slow after about two hours for high density samples with >100 clones/FOV. These results suggest that at this immobilizing agent concentration, intercolony competitive effects may occur and influence observed growth rates even though clones remain discretely physically resolvable by the detection system. The ability to determine the total cell density in an immobilized sample and to plot the growth rates of individual microorganisms may facilitate discrimination of competitive effects from growth responses to imposed test conditions, such as antibiotic susceptibility testing, for high density samples, thereby facilitating accurate AST performance over a wide range of sample conditions without necessitating additional time-consuming sample handling steps.

### Example 6

### Detection of Cross-Species Interference in Immobilized Format—Toxin Diffusion

### Background

Bacteria and fungi are known to produce small molecule compounds as well as peptides and proteins, which can inhibit other species in circumstances where both are competing for nutrients. These generally fall into one of several classes of antibiotics but may also be molecules that absorb nutrients such that they can only be used by the species that produces the compound. Some known examples of this phenomenon come from the genus Pseudomonas which produces small molecule toxins including a class that acts against other members of the same species (bacteriocin) as well as a class of iron scavenging molecules that bind to species-specific receptors for uptake (siderophore).

Generally, methods for detecting cross-species toxicity involve liquid or plate-based co-cultures of the organisms looking for differences between growth in the co-culture versus cultures of the single organisms in bulk growth assays or end point assays (i.e., macroscopic assessment of plate-based cocultures). Cell-free post-culture media or purified components from the toxin-producing organism can also be used to determine the effects of soluble factors on growth of a test organism. Analytical methods such as chromatography or mass-spectroscopy can be used to detect the presence of the toxin once it has been characterized. However, no methods exist to determine the effects of cross-species toxicity in the absence of other information such as having a sentinel organism that is known to be susceptible to the inhibitor.

When samples that contain two or more unknown species are analyzed for antibiotic susceptibility, it is important to understand the effects of the bacteria on each other. A method that is capable of examining the effects of substances produced by one unknown organism on another unknown organism would be desirable for detection of such an effect in multi-species cultures. Agar-based media are known to allow diffusion rate-based toxicity effects such as in the disk-diffusion antibiotic susceptibility test. The agar acts to limit the diffusion of larger molecules more than it does for smaller molecules, resulting in different inhibition zone sizes for different classes of antibiotics. Immobilization of the sample in the presence of a diffusion-limiting medium with analysis of the growth of the two species with respect to colony distance can provide information about cross-species inhibition and may allow rough determination of the size or class of the inhibitor based on the inhibition distance in a given medium.

### Method

A microorganism known to produce a small molecule toxin is obtained for testing as the effector. Once such effector organism is Pseudomonas fluorescens ATCC 49323, which produces the small molecule toxin mupirocin. A second microorganism known to be sensitive to the toxin produced by the first organism is obtained as a sentinel microorganism. Once such sentinel microorganism for the effects of mupirocin is Staphylococcus aureus ATCC 25923. Separate cultures of each microorganism are prepared prior to immobilization such that they can be mixed in various concentrations and at different ratios to examine the cross-species toxin effect. The samples of each single microorganism as well as the different concentrations and ratios are immobilized in a diffusion-limiting immobilizing medium. The sample medium is introduced to a biosensor device comprising microchannel flowcells in a pre-immobilization form containing the bacteria, or the pre-immobilization medium is overlaid on surface-captured bacteria. Once the pre-immobilization sample is introduced into a flowcell chamber, it is cooled to solidify and immobilize the bacteria for growth analysis. Using time-lapse microscopy, clones of each species are differentiated in early stages of growth based on clone morphology and/or identification testing, and the rate of growth is determined based changes in cell number, colony size, and/or colony brightness over time.

In this example, the two microorganism species provided in the various samples show different mean distances between the effector and sentinel colonies during growth. Samples that show very close association of the effector and sentinel will show greater inhibition of the sentinel species than samples where the colonies are separated by greater distance. At some ratio and concentration, the growth of the sentinel will be identical to that of the sentinel alone (non-inhibited sentinel). The difference in average colony distance where an effect is observed versus the average distance for the non-inhibited sample indicates the “inhibitory distance” which can be thought of as similar to a disk-diffusion zone diameter.

In a further example, a strain of the same species as the sentinel is also used that is resistant to the toxin. For this example, a Staphylococcus aureus strain that produce the mupA gene and is known to demonstrate resistance to mupirocin is also included. Mupirocin resistant Staphylococcus aureus strain ATCC BAA-1708 is used to demonstrate smaller inhibition distances than the highly sensitive ATCC 25923 sentinel, providing evidence that the inhibition is the result of the mupirocin rather than being the result of nutrient depletion.

The advantage of an immobilized format assay is that the inhibition can be tested quickly and without the need for characterized or purified toxin. The same assay can be performed for species that produce peptide or protein antibiotics that will diffuse more slowly in a diffusion limiting immobilizing medium than in traditional assays or using agar medium. Furthermore, changes to the concentration of the immobilizing agent can allow more or less diffusion in cases where the observed inhibitory distance is too great for a sensitive sentinel or too small for a resistant one.

The preceding examples are included by way of illustration, not by way of limitation. While the examples above are described in sufficient detail to enable those skilled in the art to practice various embodiments of the present disclosure, other aspects and embodiments may be realized and changes may be made without departing from the spirit and scope of the present disclosure.

Benefits, other advantages, and solutions to problems have been described herein with regard to specific embodiments. However, the benefits, advantages, solutions to problems, and any elements that may cause any benefit, advantage, or solution to occur or become more pronounced are not to be construed as critical, required, or essential features or elements of the disclosure. The scope of the disclosure is accordingly to be limited by nothing other than the appended claims, in which reference to an element in the singular is not intended to mean “one and only one” unless explicitly so stated, but rather “one or more.” Moreover, where a phrase similar to ‘at least one of A, B, and C’ or ‘at least one of A, B, or C’ is used in the claims or specification, it is intended that the phrase be interpreted to mean that A alone may be present in an embodiment, B alone may be present in an embodiment, C alone may be present in an embodiment, or that any combination of the elements A, B and C may be present in a single embodiment; for example, A and B, A and C, B and C, or A and B and C. Although the disclosure includes a method, it is contemplated that it may be embodied as computer program instructions on a tangible, non-transitory memory or computer-readable carrier, such as a magnetic or optical memory or a magnetic or optical disk. All structural, chemical, and functional equivalents to the elements of the above-described exemplary embodiments that are known to those of ordinary skill in the art are expressly incorporated herein by reference and are intended to be encompassed by the present claims. Moreover, it is not necessary for a device or method to address each and every problem sought to be solved by the present disclosure, for it to be encompassed by the present claims. Furthermore, no element, component, or method step in the present disclosure is intended to be dedicated to the public regardless of whether the element, component, or method step is explicitly recited in the claims. No claim element herein is to be construed under “means plus function”—like claim interpretation unless such claim expressly recites using the phrase “means for.” As used herein, the terms “comprises”, “comprising”, or any other variation thereof, are intended to cover a non-exclusive inclusion, such that a process, method, article, or apparatus that comprises a list of elements does not include only those elements but may include other elements not expressly listed or inherent to such process, method, article, or apparatus.

We claim:

1. A method of immobilizing microorganisms comprising:
contacting a sample comprising a plurality of microorganisms at a sample microorganism concentration with an immobilizing agent to produce a pre-immobilization sample with a pre-immobilization sample microorganism concentration and a pre-immobilization sample composition;
immobilizing the pre-immobilization sample to produce an immobilized sample having immobilized sample properties and an immobilized sample volume;
confining a first microorganism to a first location in the immobilized sample volume in response to producing the immobilized sample; and
confining a second microorganism to a second location in the immobilized sample volume in response to producing the immobilized sample;
wherein the first location and the second location are distinguishable by a detection system configured to acquire microorganism information.

2. The method of claim 1, further comprising adjusting at least one of the pre-immobilization sample microorganism concentration and the pre-immobilization sample composition in response to the sample microorganism concentration, a sample debris concentration, and a sample composition.

3. The method of claim 1, wherein the immobilized sample properties comprise an increased resistance of the immobilized sample to particle movement within the immobilized sample.

4. The method of claim 1, further comprising inducing a phase change of the immobilizing agent in the pre-immobilization sample to produce the immobilized sample.

5. The method of claim 1, wherein the sample is biological specimen.

6. The method of claim 1, wherein the immobilized sample volume is a unitary volume.

7. The method of claim 1, wherein the immobilized sample volume is discontinuous.

8. The method of claim 6, wherein immobilizing the pre-immobilization sample comprises producing a plurality of immobilized samples from the pre-immobilization sample.

9. The method of claim 8, further comprising placing each of the plurality of immobilized samples in a condition.

10. The method of claim 5, wherein the sample comprises sample debris, and wherein at least one of the plurality of microorganisms is viable.

11. The method of claim 1, wherein the pre-immobilization sample microorganism concentration is adjusted in response to the sample microorganism concentration to produce a pre-immobilization sample microorganism concentration of between 2.5×10⁵ CFU and 2.5×10⁷ CFU per milliliter.

12. The method of claim 1, wherein the pre-immobilization sample microorganism concentration is less than or equal to a concentration that would produce a 30% physical interference rate between growing clones within a 4 hour growth period.

13. A method of immobilizing microorganisms comprising:
contacting a sample comprising a plurality of microorganisms with an immobilizing agent to produce a pre-immobilization sample;
immobilizing the immobilizing agent to produce an immobilized sample having an immobilized sample volume;
confining a first microorganism to a first location in the immobilized sample volume;
confining a second microorganism to a second location in the immobilized sample volume;
detecting the first microorganism at the first location in the immobilized sample volume;
acquiring first microorganism information in response to measurement of a microorganism attribute at the first location at a first time;
acquiring first microorganism information in response to measurement of the microorganism attribute at the first location at a second time; and
determining first microorganism growth in response to a change in microorganism information acquired at the first location between the first time and the second time.

14. The method of claim 13, wherein a second microorganism attribute associated with the second microorganism is substantially prevented from influencing the first microorganism information by the immobilizing agent.

15. The method of claim 13, wherein a change in time between the first time and the second time is one of less than about 12 hours, less than about 8 hours, less than about 6 hours, less than about 4 hours, less than about 3 hours, less than about 2 hours, less than about 1 hour, and less than about 30 minutes.

16. The method of claim 13, wherein the first microorganism undergoes one of less than about 10 doubling events, less than about 7 doubling events, less than about 5 doubling events, and less than about 4 doubling events.

17. The method of claim 13, wherein the first microorganism has a diameter of one of less than about 50 μm, less than about 25 μm, less than about 10 μm, and less than about 5 μm at the second time.

18. A method comprising:
contacting a sample comprising a plurality of microorganisms with an immobilizing agent to produce a pre-immobilization sample;
contacting the pre-immobilization sample with a biosensor defining a detection space;
immobilizing the pre-immobilization sample to produce an immobilized sample having an immobilized sample volume defined by the detection space;
confining a first microorganism to a first location in the immobilized sample volume;
positioning the biosensor at a first position relative to a detection system configured to detect microorganisms in the detection space;
detecting the first microorganism at the first location in the detection space to obtain first microorganism location information;
assigning a first location value in response to the first microorganism location information, wherein the first location value comprises a first microorganism 3D coordinate relative to the detection space;
acquiring first microorganism information at a first time in response to a first microorganism attribute;
positioning the biosensor at a second position relative to the detection system;
repositioning the biosensor at the first position;
acquiring first microorganism information at a second time in response to the first microorganism attribute; and
determining growth of the first microorganism based on a change of the first microorganism information from the first time to the second time.

19. The method of claim 18, wherein the detection system comprises an optical detection system with an objective, and wherein an objective position may be changed with respect to the first position in at least one of an x-axis direction, a y-axis direction, and a z-axis direction.

20. The method of claim 19, wherein the objective position may be changed with respect to
the first position in the z-axis direction;
wherein the detection system determines a first microorganism focal plane objective position; and
wherein the first microorganism focal plane objective position produces an optimum first microorganism focus condition.

21. The method of claim 20, wherein the objective position may be changed to a second focal plane objective position and returned to the first microorganism focal plane objective position.

22. The method of claim 21, wherein an objective aperture may be changed between a first numerical aperture and a second numerical aperture.

23. The method of claim 22, wherein the first numerical aperture is used to determine a first microorganism preliminary focal plane objective position, and wherein the second numerical aperture is used to determine the first microorganism focal plane objective position.

24. The method of claim 23, wherein at least a second microorganism preliminary focal plane objective position is determined prior to determining the first microorganism focal plane objective position.

25. The method of claim 18, wherein an image registration shift is performed between sequential images in a time-lapse series.

26. The method of claim 25, wherein the image registration shift is performed by a translation in one of a two-dimensional plane or a three-dimensional space.

27. The method of claim 18, wherein at least one of an illumination wavelength and an illumination intensity are adjusted in response to a sample parameter to compensate for at least one of a sample light scattering and a sample light absorption.

28. The method of claim 27, wherein the sample parameter is one of dynamically determined or predetermined.

29. The method of claim 18, wherein at least one of contacting the pre-immobilization sample and immobilizing the pre-immobilization sample are optimized to be suitable to reduce an incidence rate of a false negative microorganism detection event for a biological sample.

30. A microorganism immobilizing composition comprising:
an immobilizing agent at an immobilizing agent concentration; and
a nutrient medium at a nutrient medium concentration;
wherein the microorganism immobilizing composition is configured to be combined with a microorganism sample to produce a pre-immobilization sample;
wherein the pre-immobilization sample is configured to be fluidly transferrable into a microvolume detection device chamber in a pre-immobilization sample condition;
wherein the immobilizing agent is configured to undergo an inducible phase change in response to a phase change condition to provide an immobilized microorganism sample comprising an immobilizing agent network suitable to restrict microorganism movement in the immobilized microorganism sample; and
wherein the immobilized microorganism sample is compatible with microorganism detection using a detection system.

31. The microorganism immobilizing composition of claim 30, wherein the immobilizing agent concentration and the nutrient medium concentration are suitable to provide a final immobilizing agent concentration and a final nutrient concentration after combining the microorganism sample with the microorganism immobilizing composition.

32. The microorganism immobilizing composition of claim 30, wherein the pre-immobilization sample condition is a temperature between about 40° C. and about 42° C.

33. The microorganism immobilizing composition of claim 30, wherein the phase change condition is one of a change in temperature, addition of a chemical agent, and exposure to electromagnetic radiation.

34. The microorganism immobilizing composition of claim 30, wherein the immobilizing agent forms covalently bound network elements in response to the phase change condition.

35. The microorganism immobilizing composition of claim 30, wherein the immobilizing agent forms a physical aggregation of network elements that are not covalently bound in response to the phase change condition.

36. The microorganism immobilizing composition of claim 30, wherein the immobilizing agent is suitable to create a first microenvironment in association with a first immobilized microorganism and a second microenvironment in association with a second immobilized microorganism.

37. The microorganism immobilizing composition of claim 36, wherein the first microenvironment and the second microenvironment are not in communication with respect to at least one of a microorganism, vesicle, macromolecular sample debris particle, nucleic acid, protein, oligopeptide, virulence factor, signal molecule, exotoxin, and metabolic waste product.

38. The microorganism immobilizing composition of claim 30, wherein the immobilizing agent is agar and wherein the immobilizing agent concentration is 5 percent, wherein the nutrient medium is Mueller-Hinton Broth, and wherein the nutrient medium concentration is 1×.

39. The microorganism immobilizing composition of claim 30, wherein the immobilizing agent is agar and wherein the immobilizing agent concentration is 5 percent, wherein the nutrient medium is Mueller-Hinton Broth, and wherein the nutrient medium concentration is 5×.

40. The microorganism immobilizing composition of claim 36, wherein the microorganism immobilizing composition is at a composition temperature suitable to be fluidly combined with the microorganism sample, and wherein the agar solidifies after:
combining a volume of the microorganism immobilizing composition with four volumes of the microorganism sample to produce an immobilized microorganism sample having a final immobilizing agent concentration of 1 percent and a final nutrient concentration of 0.2×; and
cooling the immobilized microorganism sample to ambient temperature.

41. A method of detecting growth of a plurality of microorganisms comprising:
contacting a sample comprising a plurality of microorganisms with a detection device;
immobilizing the plurality of microorganisms with an immobilizing medium, wherein immobilizing comprises confining a first microorganism in a first location defined by a physical boundary;
acquiring first microorganism information for at least the first microorganism at a first time;
acquiring second microorganism information for at least the first microorganism at a second time; and
detecting growth of the first microorganism based on first microorganism information and second microorganism information.

42. The method of claim 41, wherein the plurality of microorganisms is immobilized in a substantially planar space.

43. The method of claim 41, wherein the plurality of microorganisms is immobilized in a three-dimensional space.

44. The method of claim 41, wherein at least a portion of the physical boundary defining the first location comprises an immobilizing agent having material properties.

45. The method of claim 44, wherein the material properties of the immobilizing agent do not substantially affect one of homeostasis and a growth rate of the first microorganism.

46. The method of claim 41, wherein the physical boundary permits the diffusion of at least one of small molecules, nutrients, ions, and antimicrobial agents.

47. The method of claim 41, wherein the physical boundary produces a discrete microenvironment suitable to restrict diffusion of at least one of a secreted protein, extracellular protein, glycoprotein, enzyme, virulence factor, exotoxin, nucleic acid, vesicle, and macromolecular structure to or from an adjacent second location.

48. The method of claim 41, wherein the immobilizing medium comprises a gelling agent suitable to provide one of a polymer network and a colloidal network.
